SEQUENCE LISTING (18 sequences)

SEQ ID NO: 1 - DNA, 26 bp, Artificial
  Forward primer for human GLUT1
ggggcatgattggctccttctctgtg

SEQ ID NO: 2 - DNA, 26 bp, Artificial
  Reverse primer for human GLUT1
aggccgcagtacacaccgatgatgaa

SEQ ID NO: 3 - DNA, 26 bp, Artificial
  Forward primer for human GLUT3
tgtggtcctatgccgaatgccctcag

SEQ ID NO: 4 - DNA, 26 bp, Artificial
  Reverse primer for human GLUT3
gcaccaagaagggaaagggagactga

SEQ ID NO: 5 - DNA, 26 bp, Artificial
  Forward primer for human GLUT5
cccgagtcaattaaacagctggtcct

SEQ ID NO: 6 - DNA, 26 bp, Artificial
  Reverse primer for human GLUT5
ggaagacctgttggggccaccgagtt

SEQ ID NO: 7 - DNA, 26 bp, Artificial
  Forward primer for human LAT1
tgggacgtggacatgcctcaaggata

SEQ ID NO: 8 - DNA, 26 bp, Artificial
  Reverse primer for human LAT1
gggcacacagcaccaaggatcacaga

SEQ ID NO: 9 - DNA, 26 bp, Artificial
  Forward primer for human ENT1
ctgagtggtctggcggttttttcttc

SEQ ID NO: 10 - DNA, 26 bp, Artificial
  Reverse primer for human ENT1
acgcagacacacagacacatacacag

SEQ ID NO: 11 - DNA, 26 bp, Artificial
  Forward primer for human SMVT
cgaccttggttccctgagttctctga

SEQ ID NO: 12 - DNA, 26 bp, Artificial
  Reverse primer for human SMVT
ccaacaggcccacaacagcaccagga

SEQ ID NO: 13 - protein, 21 aa, Homo sapiens
ASQSDKTPEELFHPLGADSQV

SEQ ID NO: 14 - protein, 35 aa, Homo sapiens
TRAFEGQAHGADRSGKDGVMEMNSIEPAKETTTNV

SEQ ID NO: 15 - protein, 30 aa, Homo sapiens
NQIFTKMNKVSEVYPEKEELKELPPVTSEQ

SEQ ID NO: 16 - protein, 45 aa, Homo sapiens
MAGAGPKRRALAAPAAEEKEEAREKMLAAKSADGSAPAGEGEGVT

SEQ ID NO: 17 - protein, 51 aa, Homo sapiens
QQLKLEGPGEQETKLDLISKGEEPRAGKEESGVSVSNSQPTNESHSIKAIL

SEQ ID NO: 18 - protein, 61 aa, Homo sapiens
LSCQKRLHCRSYGQDHLDTGLFPEKPRNGVLGDSRDKEAMALDGTAYQGSSSTCILQETSL

(19) United States
(12) Patent Application Publication
(10) Pub. No.: US 2007/0231260 A1
(43) Pub. Date: Oct. 4, 2007

(54) Transporter-targeted methods of diagnosis and treatment

(75) Inventors: Noa Zerangue, Sunnyvale, CA (US); William J. Dower, Menlo Park, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: XenoPort, Inc., Santa Clara, CA (US)

(21) Appl. No.: 11/529,562

(22) Filed: Sep. 27, 2006

Related U.S. Application Data
(60) Provisional application No. 60/722,189, filed on Sep. 30, 2005.

(51) Int. Cl. A61K 49/00; G01N 33/50

(52) U.S. Cl. 424/9.1; 435/7.21

(57) ABSTRACT

Methods of treating a disease in a patient, methods of determining the presence of a disease in a patient, methods of determining whether a disease in a patient is suitable to be treated with a therapeutic agent, and methods of monitoring treatment of a disease in a patient comprising determining a level of expression of a transporter in cells of a tissue associated with the disease are disclosed. The methods include administering to a patient a diagnostic conjugate and/or therapeutic conjugate that are substrates for a transporter expressed by cells of a tissue associated with the disease. Kits comprising a diagnostic composition comprising a diagnostic conjugate that is a transporter substrate are also disclosed. In particular, methods and kits useful for diagnosing and treating cancer are disclosed.

### CROSS REFERENCES TO RELATED APPLICATIONS

This application claims benefit of U.S. Provisional Application No. 60/722,189 filed Sep. 30, 2005, which is incorporated by reference herein in its entirety.

### FIELD OF THE INVENTION

This disclosure relates to methods of treating a disease in a patient, methods of determining the presence of a disease in a patient, methods of determining whether a disease in a patient is suitable to be treated with a therapeutic agent, and methods of monitoring treatment of a disease in a patient comprising determining a level of expression of a transporter in cells of a tissue associated with the disease. This disclosure also relates to methods that include administering to a patient a diagnostic conjugate and/or therapeutic conjugate that are substrates for a transporter expressed by cells of a tissue associated with the disease. This disclosure further relates to kits comprising a diagnostic composition comprising a diagnostic conjugate that is a transporter substrate are also disclosed. In particular, this disclosure relates to methods and kits useful for diagnosing and treating cancer.

### BACKGROUND OF THE INVENTION

Cancer remains the second leading cause of death in the developed world, with solid tumors of the lung, colon, breast, prostate, pancreas, ovary, and testis accounting for the majority of cancer deaths. Cancer mortality rates for solid tumors have remained largely unchanged despite the many advances in understanding how solid tumors arise, diagnostic screening, and new cancer drugs.

Small molecule chemotherapeutics such as anti-metabolites and DNA damaging agents typically do not result in a cure for solid tumor cancer, but have clinical value in slowing disease progression and are an important component of cancer therapy due to their efficacy against a broad range of tumor types and their ability to penetrate solid tumors. These drugs target rapidly dividing malignant cells, halting cell proliferation by interfering with DNA replication, cytoskeletal rearrangements, and/or signaling pathways that promote cell growth. Disruption of cell division not only slows growth of malignant cells but can also kill tumor cells by triggering cell death. Unfortunately, these drugs also kill normal populations of proliferating cells such as those in the immune system and gastrointestinal tract, causing strong deleterious side effects, such as organ failure, that can severely limit tolerated doses and compromise effectiveness.

Thus, many chemotherapy drugs suffer from poor therapeutic indices and low overall efficacy. Although many side effects such as nausea or anemia can be addressed with palliative treatments, such toxicities often preclude the use of doses of chemotherapeutics required to better control tumor growth. In addition, side effects can negatively impact patient quality of life. Furthermore, although many chemotherapeutics have proven clinical efficacy, malignant cancers generally become resistant to antineoplastic chemotherapy. Chemotherapeutic agents that are more selectively targeted to tumor cells may exhibit lower systemic toxicity and allow more aggressive and effective treatment regimens.

Unless actively transported into a tumor cell, small molecule anti-cancer drugs rely on passive diffusion to gain access to the cytoplasm of tumor cells. Passively absorbed drugs have the dual disadvantages of ready absorption by normal tissues and inadequate penetration into poorly vascularized regions of solid tumors (Gillies et al., Neoplasia 1999, 3, 197-207). In addition, xenobiotic efflux pumps such as P-glycoprotein, MRP1, and the breast cancer resistance pump can prevent drug accumulation in tumor cells, particularly with drugs that are lipophilic or amphipathic and which are more readily passively absorbed (Longley and Johnston, J. Pathol. 2005, 275-92). These mechanisms combine to limit exposure and efficacy of anticancer drugs to tumor cells. Effective tumor penetration appears to occur with drugs that are water soluble and actively transported into tumor cells, such as anti-metabolites and contrast reagents used for diagnostic imaging.

Nutrient molecules such as amino acids, sugars, and vitamins are hydrophilic and do not readily cross lipid membranes, such as the cellular plasma membrane (Hediger et al., Eur. J. Physiol. 2004, 447, 465-469). Active transporters situated in the plasma membrane are required for uptake of these nutrient molecules into cells. Since tumor cells require energy sources for growth and chemical building blocks not synthesized by the tumors themselves, it is axiomatic that transporters for many essential nutrients are expressed in tumor cells. Furthermore, tumors are often poorly vascularized, and as a consequence some active transport systems may be up regulated or overexpressed to concentrate scarce nutrients (Bhujwall et al., Novartis Foundation Symposium 2001, 240, 23-48). Not only do tumor cells require nutrients for growth, there is also evidence that tumor energy metabolism is inefficient, especially in hypoxic solid tumors, which can result in a greater demand in tumor tissues for energy sources such as glucose and glutamine (Gatenby and Gillies, Nat. Rev. Cancer 2004, 4, 891-99). Thus, it is reasonable to premise the presence of elevated levels of expression of active nutrient and energy source transporters in tumor cells, and in particular tumor cells associated with hypoxic solid tumors. The elevated levels of transporter expression in tumor cells disclosed infra support this conclusion.

Tumor-selective nutrient accumulation is most clearly evident in imaging studies of human tumors using positron emission tomography (PET). The most widely used imaging reagent for PET is fluorodeoxyglucose (FDG) (Phelps, J. Nucl. Med. 2000, 41, 661-81). FDG accumulates at high levels in many kinds of solid tumors and has proven useful for detecting the presence of many types of solid tumors. FDG is thought to be taken up into tumor cells by sugar transporters such as facilitative glucose transporters that are highly expressed in tumor cells and quickly trapped in tumor cells by phosphorylation (Smith, British J. Biomed. Sci. 1999, 57, 170-78). There is extensive literature demonstrating expression of the facilitative glucose transporters GLUT1, GLUT3, and/or GLUT5 in certain tumor types (Smith, British J. Biomed. Sci. 1999, 56, 285-92). The level of GLUT1 expression also appears to be strongly correlated with patient prognosis, e.g., high GLUT1 expression generally indicating more advanced cancer and reduced life expectancy.

A recognized drawback of using FDG for tumor imaging is that certain pathological and non-pathological tissue, such as inflamed tissue, can also show elevated FDG accumulation, resulting in potential false positive readings (Zhuang et al., Radiol. Clin. North Am. 2005, 43, 121-34). To avoid this problem, other types of nutrients have recently been evaluated for use in PET tumor diagnosis. Aromatic amino acids such as tyrosine, tyrosine analogs, and methionine exhibit significant tumor-specific accumulation in a variety of human cancers (de Boer et al., J. Nucl. Med. 2004, 45, 2052-57; Hustinx, J. Nucl. Med. 2003, 44, 533-539; Van Laere, Eur. J. Nucl. Med. Mol. Imaging 2005, 32, 39-51). Also the monocarboxylate compound, acetate, shows clinically significant tumor specific accumulation (Shoder and Larson, Semin. Nucl. Med. 2004, 34, 274-92). Other imaging agents that exhibit tumor selectivity in clinical imaging trials include nucleoside analogs and choline (Id.; Frances et al., Eur. J. Nucl. Med. Mol. Imaging 2004, 31, 928-39). These studies provide strong evidence that tumors express amino acid, nucleoside, monocarboxylate, and vitamin transporters at high levels, and that use of these transport systems for directed uptake of tumor-targeted diagnostics and therapeutics can result in significant tumor specificity.

Several chemotherapy drugs in clinical use are actively transported into tumor cells. The best-studied examples are antimetabolites. Nucleoside analogs such as gemcitabine and 5-fluorouridine have negligible passive plasma membrane permeability but are taken up into tumor cells by transport proteins specific for nucleosides (Rauchwerger et al., Cancer Res. 2000, 60, 6075-79). The equilibrative nucleoside transporters, ENT1 and ENT2, are expressed in some tumors (Farre et al., Int. J. Cancer 2004, 112, 959-966). In certain cancer cell lines, active nucleoside transport is believed to contribute to the favorable therapeutic index of these nucleoside chemotherapeutics relative to other chemotherapeutics. The folate analogue, methotrexate, is also actively transported into tumor cells by the reduced folate carrier (RFC1) (Moscow et al., Leuk. Lymphoma 1998, 30, 215-24).

In addition, a number of alkylating agents may be actively transported into cancer cells. The nitrogen-mustard containing compound, melphalan, resembles an aromatic amino acid. It has been suggested that this compound is actively transported by the large neutral amino acid transport system (LNAA) (Uchino et al., Mol. Pharmacol. 2002, 61, 729-37). A sugar compound, D-19575 (glufosfamide), containing a phosphoramide mustard moiety is being tested in clinical trials (Briasoulis et al., J. Clin. Oncol. 2000, 18, 3535-44; Veyhll et al., Proc. Natl. Acad. Sci. 1998, 95, 2914-19; and Haberkorn et al., Magn. Reson. Med. 1998, 39, 754-61). In vitro studies suggest the involvement of sugar transport proteins. Preclinical studies suggest that D-19575 exhibits enhanced tumor accumulation relative to normal tissues when compared with the passively absorbed drug isofosfamide, which contains a similar alkylating group. It is not clear if the overexpression of transporters such as GLUT1 and GLUT3 is involved with the cellular uptake of glufosfamide or whether an active mustard species is released in plasma by a glucosidase.

Another example of an antineoplastic agent with high transport selectivity is the compound streptozotocin, a sugar molecule containing a nitrosourea alkylating group (Elsner et al., Diabetologia 2000, 43, 1528-33). When injected into rodents, streptozotocin destroys the islet beta cells of the pancreas resulting in insulin-dependent diabetes. Streptozotocin has been shown to be a selective substrate for the facilitative glucose transporter, GLUT2, which is highly expressed in pancreatic beta cells. Cell lines over-expressing GLUT2 exhibit nearly a 1000-fold greater sensitivity, e.g., cytotoxicity, to streptozotocin. Therefore, an alkylating agent that is selectively recognized by a specific transporter can exhibit a high degree of antineoplastic specificity.

The recognition that the sodium/iodide symporter (NIS) mediates the active transport of iodine and is overexpressed in thyroid cancer as well as in other tissues has enabled the extension of radioiodide therapy to extrathyroidal tissues (see e.g., Dohan et al., Endocrine Reviews, 2003, 24(1), 48-77; Robbins and Schlumerger, J Nucl Med 46(Suppl. 1), 28S-37S; Spitzweg and Morris, Clin Endocrinol 2002, 57(5), 559-74; and Heufelder et al., Thyroid 2001, 11(9), 839-47). It has recently been shown that NIS is endogenously functionally expressing in breast cancer and is functionally expressed in otherwise non NIS-expressing cancers by the ectopic transfer of the NIS gene (Doahn and Carrasco, Molecular and Cellular Endocrinology 2003, 213, 59-70). The use of NIS expression for the diagnosis and treatment of breast cancer and other extrathyroidal cancers is an active area of current research.

Although the potential of active transport strategies for increasing drug uptake into tumor cells is known, and active transport of chemotherapeutics recognized as an important uptake mechanisms, chemotherapeutics have not been optimized for transport rate or selectivity against transporters with known expression in tumor cells. Furthermore, many chemotherapeutics exhibit low response rates, most below 30%. Many transporters are overexpressed in tumors, and for many transporters there appears to be a subset of patients with much higher mRNA or protein overexpression. This subset of patients is expected to exhibit even greater tumor-specific accumulation of transported cytotoxics, and may preferentially benefit from transporter targeted therapies. Whereas many targeted therapies select patients using biopsy samples, non-invasive diagnostic methods, such as for example positron emission tomography, could be a valuable tool to assess functional tumor transporter levels and to identify the patients most likely to respond to transporter targeted chemotherapeutics.

### SUMMARY OF THE INVENTION

Despite the recognition that transporters are highly expressed and/or over expressed in certain cancers, and that active transport may underlie the efficacy of certain therapeutic agents, transporter expression has not been used as a basis for treating diseases and/or selecting therapeutic agents for treating diseases. Diagnostic and therapeutic agents that are substrates for transporters expressed in diseased tissue can be identified and modified through the use of screening methods such as, for example, the methods disclosed in U.S. Application Publication Nos. 2005/0170390; 2005/0170391; 2005/0170392; 2005/0170393; 2005/0170394; 2005/0201931; 2005/0282205; 2006/0003361; 2006/0003362; 2006/0003363; 2006/0003364; and 2006/0003920; and U.S. Provisional Application Nos. 60/703,564; 60/703,571; 60/703,572; 60/703,643; 60/703,710; and 60/703,718 filed Jul. 25, 2005. Such transporter-targeted agents can be used to characterize transporter expression in tissue and to direct treatment using transporter-targeted therapeutic agents

Certain embodiments provided by the present disclosure provide methods of treating a disease in a patient, cells of a tissue of the patient associated with the disease having a variable expression profile for a transporter across a population of patients having the disease, the method comprising screening the cells of the tissue to determine whether the cells express the transporter at an expression level above a predetermined level; if the cells express the transporter above the predetermined level, then selecting a therapeutic agent from a plurality of therapeutic agents, each of the plurality of therapeutic agents being therapeutically effective for treating the disease, the selected therapeutic agent also being a substrate for the transporter; and administering a therapeutically effective amount of the selected therapeutic agent to the patient.

Certain embodiments provided by the present disclosure provide methods of determining an expression level of a transporter in cells of a tissue of a patient associated with a disease, the cells of the tissue associated with the disease having a variable expression profile for the transporter across a population of patients having the disease, the methods comprising administering a diagnostic conjugate to the patient, wherein the diagnostic conjugate is a substrate for the transporter; measuring the uptake of the diagnostic agent into the cells of the tissue; and evaluating the measured uptake of the diagnostic conjugate to determine the expression level of the transporter in the cells of the tissue.

Certain embodiments provided by the present disclosure provide methods of determining an expression level of a transporter in cells of a first tissue associated with a disease in a patient, the methods comprising administering a diagnostic agent to the patient, wherein the diagnostic agent is a substrate for the transporter, measuring transport of the diagnostic agent through the cells of the first tissue, and evaluating the measured transport of the diagnostic agent to determine the expression level of the transporter in the cells of the first tissue after administering the diagnostic agent to the patient.

Certain embodiments provided by the present disclosure provide methods of determining the presence of a disease in a patient, the disease being characterized by an atypical level of expression for a transporter in cells of a first tissue of the patient associated with the disease, the methods comprising measuring a first expression level of the transporter in cells of the first tissue of the patient; and comparing the first expression level to a second expression level of the transporter, the second expression level being selected from: (i) an expression level of the transporter measured in cells of a second tissue of the patient that is known to be free of the disease; (ii) an expression level of the transporter measured in cells of a second tissue of the patient that is known to be unaffected by the presence of the disease; (iii) an expression level of the transporter measured in cells of a tissue of another patient who is free of the disease; and (iv) an expression level derived from a multiplicity of patients who are free of the disease.

Certain embodiments provided by the present disclosure provide methods of determining whether a disease in a patient is suitable to be treated with a therapeutic agent that is a substrate for a transporter, cells of a tissue of the patient associated with the disease having a variable expression profile for the transporter across a population of patients having the disease, the method comprising measuring an expression level of the transporter in cells of a tissue of the patient known to be associated with the disease, comparing the measured expression level of the transporter with a minimum predetermined expression level known to be useful for the therapeutic agent to be effective in treating the disease, and determining that the disease is suitable to be treated with the therapeutic agent if the measured expression level is at least the minimum predetermined expression level.

Certain embodiments provided by the present disclosure provide methods of monitoring treatment of a disease in a patient, the disease being characterized by an atypical expression level for a transporter in cells of a tissue associated with the disease, the method comprising measuring a first expression level in cells of the tissue of the patient associated with the disease; measuring a second expression level in the cells of the tissue of the patient associated with the disease, the second expression level being measured after the measuring of the first expression level and after a regimen of the treatment; and comparing the second expression level with (i) or a combination of (i) and at least one of (ii)(a), (ii)(b), (ii)(c), and (ii)(d): (i) the first expression level; and (ii)(a) an expression level of the transporter measured in cells of a second tissue of the patient that is known to be free of the disease; (ii)(b) an expression level of the transporter measured in cells of a second tissue of the patient that is known to be unaffected by the presence of the disease; (ii)(c) an expression level of the transporter measured in cells of a tissue of another patient who is free of the disease; and (ii)(d) an expression level of the transporter derived from a multiplicity of patients who are free of the disease.

Certain embodiments provided by the present disclosure provide kits comprising a first composition comprising a diagnostic conjugate and a pharmaceutically acceptable vehicle, the diagnostic conjugate being a substrate for at least one transporter expressed by cells of a tissue of the patient associated with a disease; and instructions for administering the first composition to the patient in a manner and in an amount sufficient to determine if the cells of the tissue of the patient associated with the disease characterized are by at least a minimum predetermined expression level of the at least one transporter.

Certain embodiments provided by the present disclosure provide diagnostic kits for selecting a therapeutic agent for treating a disease in a patient, cells of a tissue associated with the disease having a variable expression profile for a first transporter across a population of patients having the disease, the diagnostic kit comprising a first diagnostic composition comprising a first diagnostic agent that is a substrate for a first transporter and a pharmaceutically acceptable vehicle; a second diagnostic composition comprising a second diagnostic agent that is a substrate for a second transporter and a pharmaceutically acceptable vehicle; and instructions for administering a diagnostically effective amount of the first and second diagnostic compositions to the patient for measuring the expression profiles of the first and second transporters in the tissue of the patient associated with the disease, and for selecting an appropriate therapeutic agent for treating the disease based upon the measured expression profiles of the first and second transporters.

### DESCRIPTION OF THE DRAWINGS

FIG. 1 shows uptake of bromopyruvate in cells not induced (−TET) to express MCT1 and in cells induced (+TET) to express MCT1.

FIG. 2 shows the cytotoxicity, GI50, of bromopyruvate in cells not expressing MCT1 (−TET) and in cells induced to express MCT1 (+TET).

FIG. 3 shows the correlation between the GI50 for bromopyruvate and the level of MCT1 expression in cancer cell lines.

FIG. 4 shows the dose dependent inhibition of SMVT transport by the phosphoramide prodrug 4-({(acetyloxymethoxy)[bis(2-chloroethyl)amino]carbonyl}amino)butanoic acid (1).

FIG. 5 shows the SMVT-dependent cytotoxicity of compound (1) in HEK cells.

FIG. 6 shows inhibition of growth of HP69 tumors in a mouse xenograph tumor model by compound (1).

### DEFINITIONS

“Transport by passive diffusion” refers to transport of an agent not mediated by a specific transporter protein. An agent substantially incapable of passive diffusion has a permeability across a standard cell monolayer (e.g., Caco-2 or MDCK cells or an artificial bilayer (PAMPA)) of less than 5×10⁻⁶ cm/sec, and usually less than 1×10⁻⁶ cm/sec in the absence of an efflux mechanism.

A “substrate” of a transport protein, e.g. a transporter, is a compound in which uptake into a cell or translocation through the plasma membrane of a cell is facilitated, at least in part, by the transporter protein.

“Diagnostic agent” refers to a compound that has diagnostic activity. Diagnostic agents include compounds that are known to be diagnostic agents, compounds that are identified as having diagnostic activity and are undergoing further diagnostic evaluation, and compounds that are members of collections and libraries that are to be screened for a diagnostic activity. Diagnostic agents include compounds that can be used for in vivo imaging following administration to a patient, and that can be imaged by methods such as, but not limited to, optical methods, radiography, positron emission tomography, single photon emission computer-aided tomography, and magnetic resonance.

“Therapeutic agent” refers to a compound that has or may have a pharmacological activity. Agents include compounds that are known drugs, compounds that are identified as having pharmacological activity and are undergoing further therapeutic evaluation, and compounds that are members of collections and libraries that are to be screened for a pharmacological activity. Therapeutic agents can have demonstrated or potential efficacy for treating a disease in a patient.

“Conjugate” refers to a compound comprising an agent and a chemical substrate moiety bound thereto, which moiety by itself or in combination with the agent renders the conjugate a substrate for transport through a cell plasma membrane, for example rendering the conjugate to be a substrate for a transport protein. The substrate moiety may or may not be subject to cleavage from the agent upon uptake and metabolism of the conjugate in a patient's body. In other words, the substrate moiety can be cleavably bound to the agent or non-cleavably bound to the agent. The bond between an agent and a substrate moiety can be a direct bond, i.e., a covalent bond, or the bond can be through a linker. In cases where the bond/linker is cleavable by metabolic processes, the agent, or a further metabolite of the agent, can be a diagnostic or therapeutic entity. In cases where the bond/linker is not cleavable by metabolic processes, the conjugate can be a diagnostic or therapeutic entity. Conjugates can comprise a prodrug having a metabolically cleavable substrate moiety, where the conjugate itself does not have pharmacological activity but the agent to which the substrate moiety is cleavably bound exhibits pharmacological activity. Typically, a substrate moiety facilitates therapeutic use of the agent by promoting cellular uptake of the conjugate via a transporter protein. Thus, for example, a conjugate comprising an agent and a substrate moiety may have a Vₘₐₓ for a transporter at least 2, 5, 10, 20, 50, or 100-fold higher than that of the agent alone. A substrate moiety can itself be a substrate for a transporter protein or can become a substrate when linked to an agent, e.g., valacyclovir, an L-valine ester prodrug of the antiviral drug acyclovir. Thus, a conjugate formed from an agent and a substrate moiety can have higher uptake activity than either the agent or the substrate moiety alone.

“Cancer,” “cancerous tissue,” or “cancerous cell” refers to a tissue or cell that has lost or partially lost the ability to control cell division. A cancerous cell can be a cell line such as HeLa, MOLT4, and others, and can also be a cell obtained from a patient. A cancerous cell from a patient can be from a solid tumor such as a tumor of the colon or from a non-solid tissue such as blood, e.g., leukemia. A cancerous cell can be isolated from a human or animal, such as cells obtained from a tissue biopsy. Alternatively, a cancer cell can be present in a human or animal. Certain cancerous tissue can comprise tumors.

Malignant cancers are those that invade surrounding tissues and metastasize to other body sites via the systemic circulation. Metastasized cancers usually remain the same type of cell as the initial site of cancer development. For example, if breast cancer metastasizes to a lung, the cancer in the lung consists of breast cells. Benign cancers do not invade other tissues or spread, have a slower growth rate than malignant cancers, and in most cases are not fatal.

“Diagnostically effective amount” means the amount of a compound that, when administered to a patient for diagnosing a disease or disorder, is sufficient to affect such diagnosis of the disease or disorder. A “diagnostically effective amount” can vary depending, for example, on the compound, the disease or disorder and its severity, and the age, weight, etc., of the patient to which the compound is administered. A diagnostically effective amount also can mean the amount of a compound that, when contacted with a cell, for example, in vitro, is sufficient to affect such diagnosis.

“EC₅₀” is a measurement of the substrate concentration that results in a turnover rate that is 50% of the maximal turnover rate for the substrate (0.5 Vₘₐₓ).

“GI₅₀” refers to the concentration of a compound at 50% growth inhibition.

“Patient” includes mammals, for example humans. Patient includes those having a disease, those suspected of having a disease, and those in which the presence of a disease is being assessed.

“Pharmaceutically acceptable” refers to approved or approvable by a regulatory agency of a federal or a state government, or listed in the U.S. Pharmacopoeia or listed in other generally recognized pharmacopoeia for use in mammals, including humans.

“Pharmaceutical composition” refers to at least one compound such as a diagnostic agent or therapeutic agent, and a pharmaceutically acceptable vehicle with which the compound is administered to a patient.

“Pharmacological activity” means that a therapeutic agent exhibits an activity in a screening system, which is or may be useful in the treatment of a disease. The screening system can be in vivo, in vitro, cellular, animal, and/or human. Therapeutic agents can be described as having pharmacological activity notwithstanding that further testing may be required to establish actual therapeutic utility in treating a disease.

“Substrate moiety” refers to an entity that imparts substrate activity to a conjugate, as defined herein.

“Sustained release” refers to release of a therapeutic amount of a drug or an active metabolite thereof over a period of time that is longer than a conventional formulation of the drug. For oral formulations, the term “sustained release” typically means release of the drug within the gastrointestinal tract lumen over a period ranging from about 2 to about 30 hours, and in certain embodiments, over a period ranging from about 4 to about 24 hours. Sustained release formulations achieve therapeutically effective concentrations of the drug in the systemic circulation over a prolonged period of time relative to that achieved by oral administration of an immediate release formulation of the drug. “Delayed release” refers to release of the drug or an active metabolite thereof into the gastrointestinal lumen after a delay time period, for example a delay of about 1 to about 12 hours, relative to that achieved by oral administration of an immediate release formulation of the drug. In some embodiments, sustained release refers to release of a diagnostic amount of a diagnostic agent or an active metabolite thereof over a period of time that is longer than an immediate release formulation of the diagnostic agent.

“Therapeutically effective amount” refers to the amount of a compound that, when administered to a subject for treating a disease, or disorder, or at least one of the clinical symptoms of a disease or disorder, is sufficient to affect such treatment of the disease, disorder, or symptom. The “therapeutically effective amount” may vary depending, for example, on the compound, the disease, disorder, and/or symptoms of the disease, severity of the disease, disorder, and/or symptoms of the disease, the age, weight, and/or health of the patient to be treated, and the judgment of the prescribing physician. An appropriate amount in any given instance may be readily ascertained by those skilled in the art or capable of determination by routine experimentation.

“Treating” or “treatment” of a disease refers to arresting or ameliorating a disease, disorder, or at least one of the clinical symptoms of a disease or disorder. In certain embodiments, “treating” or “treatment” refers to arresting or ameliorating at least one physical parameter of the disease or disorder, which may or may not be discernible by the patient. In certain embodiments, “treating” or “treatment” refers to inhibiting or controlling the disease or disorder, either physically (e.g., stabilization of a discernible symptom), physiologically (e.g., stabilization of a physical parameter), or both. In certain embodiments, “treating” or “treatment” refers to delaying, in some cases indefinitely, the onset of a disease or disorder.

“Vₘₐₓ” and “Kₘ” of a compound for a transporter protein are defined in accordance with convention. Vₘₐₓ is the number of molecules of compound transported per second at saturating concentration of the compound. Kₘ is the concentration of the compound at which the compound is transported at half of Vₘₐₓ. When the objective is to transport an agent or conjugate into a cancer cell, a high Vₘₐₓ for an influx transporter is generally desirable. Likewise for the same objective, a low value of Kₘ can be desirable for transport of a compound present at low concentration in the systemic circulation. In some embodiments, a high value of Kₘ is acceptable for the transport of compounds present at high concentrations in the systemic circulation. For these reasons, the intrinsic capacity of a compound to be transported by a particular transporter is usually expressed as the ratio Vₘₐₓ of the compound/Vₘₐₓ of a reference compound, wherein the reference compound is known to be a substrate for the transporter. Vₘₐₓ is affected by both the intrinsic turnover rate of a transporter (molecules/transporter protein) and transporter density in the plasma membrane, which depends on the expression level of the transporter.

For sequence comparison, typically one sequence acts as a reference sequence to which test sequences are compared. When using a sequence comparison algorithm, test and reference sequences are entered into a computer, sub-sequence coordinates are designated, if necessary, and sequence algorithm program parameters are designated. The percent sequence identity for the test sequence(s) relative to the reference sequence are then calculated using the sequence comparison algorithm with the designated program parameters.

Optimal alignment of sequences for comparison can be conducted, for example, using the local homology algorithm of Smith & Waterman, Adv. Appl. Math. 1981, 2, 482, by the homology alignment algorithm of Needleman and Wunsch, J. Mol. Biol. 1970, 48, 443, by the search for similarity method of Pearson and Lipman, Proc. Nat'l. Acad. Sci. USA 1988, 85, 2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., eds., Current Protocols, Greene Publishing Associates, Inc. and John Wiley & Sons, Inc., (1998 Supplement).).

Another example of an algorithm suitable for determining percent sequence identity and sequence similarity is the BLAST algorithm, which is described in Altschul et al., J. Mol. Biol. 1990, 215, 403-10). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction is halted when the cumulative alignment score falls off by the quantity X from its maximum achieved value, e.g., the cumulative score goes to zero or below due to the accumulation of one or more negative-scoring residue alignments, or the end of either sequence is reached. For identifying whether a nucleic acid or polypeptide is within the scope hereof, the default parameters of the BLAST programs are suitable. The BLASTN program (for nucleotide sequences) uses as defaults a word length W of 11, an expectation E of 10, M=5, N=−4, and a comparison of both strands. For amino acid sequences, the BLASTP program uses as defaults a word length W of 3, an expectation E of 10, and the BLOSUM62 scoring matrix. The TBLASTN program (using protein sequence for nucleotide sequence) uses as defaults a word length W of 3, an expectation E of 10, and a BLOSUM62 scoring matrix (see Henikoff and Henikoff, Proc. Natl. Acad. Sci. USA 1989, 89, 1915).

In addition to calculating percent sequence identity, the BLAST algorithm also performs a statistical analysis of the similarity between two sequences (see, e.g., Karlin and Altschul, Proc. Nat'l. Acad. Sci. USA 1993, 90, 5873-87). One measure of similarity provided by the BLAST algorithm is the smallest sum probability P(N), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Reference is now be made in detail to particular embodiments of methods and kits of the present disclosure. The disclosed embodiments are not intended to be limiting of the claims. To the contrary, the claims are intended to cover all alternatives, modifications, and equivalents thereof.

### DESCRIPTION OF VARIOUS EMBODIMENTS

Using appropriate screening methods, disclosed for example in U.S. Application Publication Nos. 2005/0170390; 2005/0170391; 2005/0170392; 2005/0170393; 2005/0170394; 2005/0201931; 2005/0282205; 2006/0003361; 2006/0003362; 2006/0003363; 2006/0003364; and 2006/0003920; and U.S. Provisional Application Nos. 60/703,564; 60/703,571; 60/703,572; 60/703,643; 60/703,710; and 60/703,718 filed Jul. 25, 2005 diagnostic and therapeutic agents that are transporter substrates can be identified or developed through a combination of iterative chemical synthesis and screening for transporter activity. Transporter selective agents can be used in transporter targeted diagnostic and therapeutic methods. Such methods can have particular utility in diagnosing and/or treating diseases in which tissue associated with the disease highly expresses a transporter or over-expresses a transporter relative to non-diseased tissue. For example, certain transporters are highly expressed and/or over-expressed in certain cancers, such as solid tumors. Diagnostic and therapeutic agents targeted to such transporters are expected to be particularly effective for use in diagnosing and/or treating these cancers.

Furthermore, many chemotherapeutics exhibit variable treatment efficacy and treatment response depending upon the patient and/or disease. Therefore, there is considerable interest in new diagnostic methods for identifying the subset of patients likely to benefit from a particular therapy and thereby avoid unnecessary or ineffective chemotherapy treatments. As disclosed in Examples 1 and 2, a variety of transporters are highly expressed in human colon, lung, breast ovarian, and prostate cancers. Certain transporters also exhibit a higher level of expression in tumor tissue relative to normal tissue from the same organ. The transporters GLUT1, GLUT3, GLUT5, LAT1, ENT1, MCT1, MCT4, and SMVT are examples of transporters that are highly expressed in tumors and that are differentially expressed relative to normal tissue. While many transporters are over-expressed in certain tumors, for many transporters, there appears to be a subset of patients in which the tumors exhibit a much higher level of mRNA or protein expression. This subset of patients is expected to exhibit particularly high tumor-specific accumulation of transported therapeutic agents, and therefore is expected to preferentially benefit from transporter-targeted therapies. As disclosed herein, clinical diagnostic PET imaging using transported nutrients has proven highly successful. Whereas most targeted therapies select patients using biopsy samples, PET imaging of transporter-targeted diagnostic agents can be a valuable tool to assess functional transport in diseased tissue and thereby identify those patients most likely to respond to transporter-targeted therapy.

Beyond the known therapeutic agents that are inherently substrates for a particular transporter the potential to successfully modify known therapeutic agents or to develop new therapeutic agents and conjugates that are also transporter substrates will depend in part on the capacity of a transporter to recognize and translocate molecules having a range of chemical diversity across the cellular plasma membrane. Transporters that exhibit high substrate specificity will be unlikely to recognize modified substrates such as diagnostic and therapeutic conjugates.

Several examples suggest that at least some transporters can recognize a diverse set of molecules. For example, the oligopeptide transporters PEPT1 and PEPT2 transport most naturally occurring di- and tri-peptides as well as antibiotics and amino acid nucleoside conjugates (Brodin et al., Pharmacol. Toxocol. 2000, 90, 285-96). Amino acid transporters such as LAT1 and ATB0+ recognize most natural amino acids and several amino acid drugs (Uchino et al., Mol. Pharmacol. 2002, 61, 729-37). Also, xenobiotic transporters in the liver and kidney are known to transport diverse drugs (Murer and Biber, Curr. Op. Cell. Bio. 1998, 10, 429-34).

Studies using commercially available compounds were performed to assess the range of molecules recognized by the transporters GLUT1, GLUT3, GLUT5, LAT1, SMVT, MCT1, MCT4, and ENT1, which are highly expressed in certain tumor transporters. These studies confirmed that certain tumor transporters actively transport substrates having a range of chemical structures. In certain embodiments, diagnostic agents or therapeutic agents provided by the present disclosure may be substrates for a transporter expressed by a tissue associated with a disease. Tissue associated with a disease includes tissue affected by the disease and tissue capable of mediating delivery of a diagnostic or therapeutic agent to a tissue affected by the disease. In certain embodiments, cells of a tissue affected by a disease can be targeted by a diagnostic and/or therapeutic agent, where the diagnostic and/or therapeutic agent is a substrate for a transporter expressed by the diseased tissue, and the agent can be taken up by cells of the diseased tissue via the transporter. An example of tissue affected by a disease, e.g. diseased tissue, is cancer and in particular, certain solid tumors which are shown herein to highly express the transporters GLUT1, GLUT5, LAT1, SMVT, MCT1, MCT4, and ENT1.

The family of facilitated glucose transporters (GLUTs) contains at least 14 members in humans (SLC1A1-14, GLUT1-14). GLUT transporters have 12 putative transmembrane domains with both the amino and carboxy termini located on the cytoplasmic side of the plasma membrane. Various GLUT transporters have been demonstrated to transport a variety of sugars such as glucose, 2-deoxyglucose, galactose, fructose, inositol, and sugar analogs such as dehydroascorbate, glucosamine, and fluorodeoxyglucose. Transport is bidirectional across the plasma membrane allowing transport either into or out of a cell depending on the substrate gradient. Because there is no net charge movement, transport does not depend on the transmembrane potential. The GenBank accession number for human GLUT1 is NM_{—}006516 (incorporated herein by reference). The GenBank accession number for human GLUT3 is NM_{—}006931 (incorporated by reference). The GenBank accession number for human GLUT5 is NM_{—}003039 (incorporated herein by reference).

In certain embodiments, substrates for the GLUT1, GLUT3, and GLUT5 transporters are compounds that contain 5 and 6 membered rings, and in certain embodiments, have alcohol groups attached to several of the positions on the ring. Substrates for GLUT1, GLUT3, and GLUT5 are typically sugars or vitamins. Examples of GLUT1 substrates, include, but are not limited to, glucose, galactose, dehydroascorbic acid, glucosamine, (S)-methoxy-α-methyl-2-naphthalene acetic acid amido galactopyranose, and fluorodeoxyglucose. Examples of GLUT3 substrates include, but are not limited to, glucose, glucuronic acid, dehydroascorbic acid, glucosamine, and fluorodeoxyglucose. Examples of GLUT5 substrates, include, but are not limited to, fructose and galactose. GLUT1, GLUT3, and GLUT5 are highly expressed in certain cancer cells (Examples 1-3 and Zerangue, U.S. Application Publication Nos. 2006/0003363, 2006/0003364 and 2005/0282205). Examples of cancers in which GLUT1, GLUT3, and GLUT5 are expressed at a level more than 500-fold higher than some other GLUT family transporters with similar substrate activity include breast, lung, colon, and ovarian cancer.

The family of amino acid transporter/permeases (AAPs) includes at least 13 members in humans (SLC7A1-13). AAP transporters have 12 putative transmembrane domains with both the amino and carboxy termini located on the cytoplasmic side of the plasma membrane. A sub-family of AAPs, referred to as LAT1 and LAT2, is specialized for the transport of large aromatic neutral amino acids. LAT1 and LAT2 transport a variety of neutral amino acids such as leucine, valine, isoleucine, phenylalanine, tryptophan, and methionine; and amino acid analogs such as L-dopa, gabapentin, and bicyclohexane amino acid (BCH). Both LAT1 and LAT2 co-assemble with the glycoprotein 4F2HC. LAT1 and LAT2 transporters are obligate exchange transporters. Thus, for each amino acid transported across the plasma membrane into a cell, another amino acid is effluxed out of the cell. The GenBank accession number for human LAT1 is NM_{—}003486 (incorporated herein by reference).

LAT1 is known to transport L-amino acids with bulky (>2 atoms), uncharged and hydrophobic or aromatic side chains. LAT1 also transports similar D-amino acids with lower affinity than L-amino acids. Additionally, LAT1 transports several γ-amino acids such as gabapentin and cyclic α-amino acids such as bicyclohexane amino acid. Examples of LAT1 substrates include, but are not limited to, tryptophan, leucine, L-isoleucine, methionine, phenylalanine, bicyclohexane amino acid, L-dopa, gabapentin, and baclofen. LAT1 is highly expressed in certain cancer cells (Examples 1-3 and Zerangue, U.S. Application Publication No. 2006/0003920). Examples of cancers in which LAT1 is highly expressed include breast, lung, colon, and ovarian cancer.

The family of equilibrative nucleoside transporters (ENTs) includes at least 4 members in humans (SLC29A1-SLC29A4). ENT transporters have 7-9 putative transmembrane domains. ENT1 and ENT2 transporters have been demonstrated to transport a variety of nucleoside and nucleobase compounds. Transport is bidirectional, allowing transport either into or out of a cell depending on the substrate gradients. Because there is no net charge movement, transport does not depend on the transmembrane potential. The GenBank accession number for human ENT1 is NM_{—}004955 (incorporated herein by reference).

ENT1 substrates include purine and pyrimidine nucleosides and some nucleobases such as hypoxanthine. ENT1 substrates can have a sugar group attached to a purine or pyrimidine base. Substrates of ENT1 are typically water-soluble molecules that include naturally occurring nucleosides or nucleobases or analogs thereof such as gemcitabine. Examples of ENT1 substrates include, but are not limited to, uridine, adenosine, hypoxanthine, and gemcitabine. ENT1 is highly expressed in certain cancer cells (Examples 1-3 and Zerangue, U.S. Application Publication No. 2006/0003362). Examples of cancers in which ENT1 is highly expressed include breast, lung, colon, and ovarian cancer.

SMVT (SLC5A6) is a transporter for water-soluble vitamins such as biotin and pantothenic acid. Substrate transport by SMVT has an obligatory dependence on Na⁺ ions as a co-substrate. When expressed in Xenopus oocytes, SMVT responds electrogenically (induction of an inward current) upon addition of a substrate, with the magnitude of the response being directly proportional to the rate of substrate transport. The GenBank accession number for human SMVT is NM_{—}021095 (incorporated by reference).

SMVT substrates include compounds that contain a free carboxylic acid and a short chain of 2-6 atoms ending in a cyclic or branched group. Examples of SMVT substrates include, but are not limited to, biotin, pantothenic acid, 4-phenylbutyric acid, 4-(toluene-4-sulfonylamino)-butyric acid, and N-(4-nitrobenzoyl)-β-alanine. SMVT is highly expressed in certain cancer cells (Examples 1-3 and Zerangue, U.S. Application Publication No. 2006/0003361). Examples of cancers in which SMVT is highly expressed include breast, lung, colon, and ovarian cancer.

The family of monocarboxylate transporters (MCTs) contains at least 14 members in humans (SLC16A1-14). Monocarboxylate transporters have 12 putative transmembrane domains with both the amino and carboxy termini located on the cytoplasmic side. A sub-family of monocarboxylate transporters (MCT1, MCT2, MCT4) is specialized for the transport of metabolic intermediates and cofactors such as lactic acid, pyruvic acid, and nicotinic acid, and other members (MCT8, MCT11) transport amino acids and thyroid hormones. It has been reported that MCT1 contributes to the intestinal absorption of xenobiotic drugs such as salicylic acid, pravastatin, and antibiotics. It is also reported that MCT1 co-assembles with the glycoprotein CD147. MCT1 is a proton-coupled transporter that co-transports a monocarboxylate molecule and a proton. The GenBank accession number for human MCT1 is NM_{—}003051 (incorporated herein by reference). The GenBank accession number for human MCT4 is NM_{—}0046696 (incorporated herein by reference).

Examples of MCT1 substrates include, but are not limited to, lactic acid, 4-(4-hydroxylmethyl-3-methoxyphenoxy)-butyric acid, prevastatin, and 2-thiopheneglyoxylic acid. MCT1 is highly expressed in certain cancer cell lines (Examples 1-3).

As is apparent from these examples, transporters such as GLUT1, GLUT3, GLUT5, LAT1, ENT1, MCT1, MCT4, and SMVT are highly and/or differentially expressed in certain human tumors, and are capable of transporting substrates having diverse chemical structures.

In certain embodiments, diagnostic and therapeutic agents provided by the present disclosure may be substrates for a transporter expressed by a tissue capable of mediating the delivery of a diagnostic or therapeutic agent to a target tissue. An example of a mediating tissue is brain capillary endothelial cells. Brain capillary endothelial cells are joined together by tight intercellular junctions, which form a continuous wall against the passive diffusion of molecules form the blood to the brain and other parts of the central nervous system (CNS) (Goldstein et al., Scientific American 1986, 255, 74-83; Pardridge, Endocrin. Rev. 1986, 7, 314-330). The blood-brain-barrier (BBB) formed by the brain capillary endothelial cells functions to ensure that the environment of the brain is constantly controlled despite fluctuations in the levels of various substances in the blood such as hormones and amino acids. The presence of specific transport systems within brain capillary endothelial cells assures that the brain receives all compounds necessary for normal growth and function. Several active transporters are known to be highly expressed in brain capillary endothelial cells including GLUT1, LAT1, MCT1, TAUT1, OAT3, OATPB, GAT2, OCT3, OCTN2, SVCT2, CAT1, and BGT1 (Zerangue, U.S. Application Publication Nos. 2005/01703394, 2005/0201931, 2005/0170390, 2005/0170391, 2005/0170392, and 2005/0170393; and Zerangue, U.S. Provisional Application Nos. 60/703,718, 60/703,643, 60/703,572, 60/703,564, 60/703,571, and 60/703,710 filed Jul. 29, 2005). Therapeutic agents or metabolites thereof that exhibit therapeutic activity in the treatment of a disease of the brain such as cancer, inflammation, or allergic disease; or the CNS such as neurological diseases, Acquired Immune Deficiency Syndrome, stroke, epilepsy, Parkinson's disease, multiple sclerosis, neurodegenerative disease, trauma, depression, Alzheimer's disease, migraine, pain, and cancer, and that are also substrates for any of the transporters expressed in brain capillary endothelial cells including those disclosed herein are examples of therapeutic agents that exert their effects through mediating tissue. Diseases mediated by the BBB also include those in which the diseased tissue is non-CNS tissue, but is responsive to treatment by an agent that exerts a pharmacological effect on the CNS and that in turn causes an effect on the diseased non-CNS tissue, such as an effect caused by the release of hormones in the CNS.

The GLUT1 and LAT1 transporters are disclosed, herein, within the context of examples of transporters that are highly expressed in certain solid tumors. GLUT1 is expressed in brain capillary epithelial cells at a level more than 20-fold higher than other GLUT family transporters with similar substrate specificity (Zerangue, U.S. Application Publication No. 2005/0170394). LAT1 is expressed in brain capillary endothelial cells at a level nearly 10-fold higher than other LAT family transporters with similar substrate specificity (Zerangue, U.S. Application Publication No. 2005/0201931).

The family of monocarboxylate transporters (MCTs) includes at least 14 members in humans (SLC16A1-14). Monocarboxylate transporters have 12 putative transmembrane domains, with both the amino and carboxy termini located on the cytoplasmic side. A sub-family of monocarboxylate transporters (MCT1, MCT2, MCT4) is specialized for the transport of metabolic intermediates and cofactors such as lactic acid, pyruvic acid, and nicotinic acid, whereas other members (MCT8, MCT11) transport amino acids and thyroid hormones. It has been reported that MCT1 contributes to the intestinal absorption of xenobiotic drugs such as salicylic acid, pravastatin, and antibiotics. It has been reported that MCT1 co-assembles with the glycoprotein CD147. MCT1 is proton-coupled transporter that co-transports a monocarboxylate molecule and a proton. MCT1 is expressed in brain capillary epithelial cells at a level more than 10-fold higher than other MCT family transporters with similar substrate specificity (Zerangue, U.S. Application Publication No. 2005/017390). The GenBank accession number for human MCT1 is NM_{—}003051 (incorporated herein by reference).

The family of sodium and chloride coupled neurotransmitter transporters includes at least 16 members in humans (SLC6A1-16). Neurotransmitter transporters have 10-13 putative transmembrane domains, with both the amino and carboxy termini located on the cytoplasmic side. Most neurotransmitter transporters only transport neurotransmitters and amino acids. One member of this family is TAUT1 (SLC6A6), which mediates the cellular uptake of taurine, α-alanine, and γ-aminobutyric acid (GABA). TAUT1 transport is dependent on the co-transport of sodium and chloride ions. TAUT1 is expressed in brain capillary endothelial cells at a level more than 3-fold higher than other sodium and chloride coupled neurotransmitter family transporters with similar substrate specificity (U.S. Application Publication No. 2005/0170391). The GenBank accession number for human TAUT1 is NM_{—}003043 (incorporated herein by reference).

Another member of the family of neurotransmitter transporters is GAT2 (SLC6A13), which mediates the cellular uptake of beta-alanine and GABA. GAT2 transport is dependent on the co-transport of sodium and chloride ions. GAT2 is highly expressed in brain capillary endothelial cells (Zerangue, U.S. Provisional Application No. 60/703,718). The GenBank accession number for human GAT2 is NM_{—}016615 (incorporated herein by reference).

Another member of the family of sodium and chloride coupled neurotransmitter transporters is BGT1 (SLC6A12), which mediates the cellular uptake of betaine, β-alanine, and GABA. BGT1 transport is dependent on the co-transport of sodium and chloride ions. BGT1 transport is dependent on the co-transport of sodium and chloride ions. BGT1 is highly expressed in brain capillary endothelial cells (Zerangue, U.S. Provisional Application No. 60/703,710). The GenBank accession number for human BGT1 is NM_{—}003044 (incorporated herein by reference).

The family of organic anion transporters (OATs) and organic cation transporters (OCTs) includes at least 25 members in humans (SLC21A1-12; SLC22A1-13). Organic anion and cation transporters have 12 putative transmembrane domains, with both the amino and carboxy termini located on the cytoplasmic side. A sub-family of organic anion transporters (OAT1-4) is expressed in the kidney, liver, and barrier tissues such as intestine, testis, and blood brain barrier. Members of the OAT family recognize a diverse group of organic anion compounds such as ochratoxin A, para-amino-hippurate, and estrone-3-sulphate. OAT transport is coupled to the counter transport of metabolic anions such as ketoglutarate. OAT3 is expressed in brain capillary endothelial cells at a level more than 15-fold higher than other OAT family transporters with similar substrate specificity (Zerangue, U.S. Application Publication No. 2005/0170392). The GenBank accession number for human OAT3 is NM_{—}004254 (incorporated herein by reference).

A sub-family of organic anion transporters (OATPs: OATPA, OATPB, OATPC, OATPD, OATPE, OATP8, OATP14, and PGT) is expressed in the kidney, liver, and other tissue such as intestine, testis, and the blood brain barrier. Members of the OATP sub-family recognize a diverse group of organic anion compounds such as bile acids, prostaglandins, and xenobiotics conjugated to sulfate or glucuronide. OATPB is expressed in brain capillary endothelial cells at a level more than 2.5-fold higher than other OATP family transporters with similar substrate specificity (Zerangue, U.S. Application Publication No. 2005/0170393). The GenBank accession number for human OATPB is NM_{—}007256 (incorporated herein by reference).

One member of the family of organic cation transporters is OCT3 (SLC22A3), which mediates the cellular uptake of monoamine neurotransmitters (dopamine, epinephrine), histamine, and organic cation xenobiotics such as MPP+ and quinidine. OCT3 transport is dependent on the plasma membrane potential. OCT3 is highly expressed in brain capillary endothelial cells (Zerangue, U.S. Provisional Application No. 60/703,643). The GenBank accession number for human OCT3 is NM_{—}021977 (incorporated herein by reference). Another member of the family of organic cation transporters is OCTN2 (SLC22A5), which mediates the cellular uptake of carnitine and organic cations such as tetraethylammonia. OCTN2 transport of carnitine is dependent on the co-transport of sodium ion and the membrane potentials, but transport of organic cations is sodium-independent. OCTN2 is highly expressed in brain capillary endothelial cells (Zerangue, U.S. Provisional Application No. 60/703,564). The GenBank accession number for human OCTN2 is NM_{—}003060 (incorporated herein by reference).

The family of sodium coupled ascorbic acid (vitamin C) transporters includes at least 3 members in humans (SLC23A1-3). Ascorbic acid transporters have 12 putative transmembrane domains, with both the amino and carboxy termini located on the cytoplasmic side. One member of this family is SVCT2 (SLC23A2), which mediates the cellular uptake of ascorbic acid. SVCT2 transport is dependent on the co-transport of sodium and chloride ions. SVCT2 is highly expressed in brain capillary endothelial cells (Zerangue, U.S. Provisional Application No. 60/703,564). The GenBank accession number for human SVCT2 is AF164142 (incorporated herein by reference).

The family of amino acid permease transporters includes at least 13 members in humans (SLC7A 1-13). Amino acid permease transporters have 12 putative transmembrane domains, with both the amino and carboxy termini located on the cytoplasmic side. One member of this family is CAT1 (SLC7A1), which mediates the cellular uptake of cationic amino acids such as lysine, arginine, and ornithine. CAT1 transport is dependent on the electrical plasma membrane potential. CAT1 is highly expressed in brain capillary endothelial cells (Zerangue, U.S. Provisional Application No. 60/703,571). The GenBank accession number for human CAT1 is NM_{—}003045 (incorporated herein by reference).

Unless otherwise apparent from the context, reference to a transporter includes the amino acid sequence described in or encoded by the indicated GenBank reference number and, allelic, cognate, and induced variants and fragments thereof that retain essentially the same transporter activity. Usually such variants show at least 90% sequence identity to the exemplary GenBank nucleic acid or amino acid sequence.

Known and potential diagnostic agents and therapeutic agents can be screened for transporter activity using methods known in the art. Diagnostic agents and therapeutic agents identified as transporter substrates can be iteratively modified and screened to improve the substrate properties including, for example, increasing the Vₘₐₓ and/or K_{M}, enhancing the selectivity of the agent for a particular transporter compared to other transporters, reducing the Vₘₐₓ and/or K_{M} for efflux transporters, etc. Methods for screening compounds for transporter substrate activity and iteratively modifying and testing activity for certain transporters expressed in human tumors are disclosed in U.S. Application Publication Nos. 2005/0170390; 2005/0170391; 2005/0170392; 2005/0170393; 2005/0170394; 2005/0201931; 2005/0282205; 2006/0003361; 2006/0003362; 2006/0003363; 2006/0003364; and 2006/0003920; and U.S. Provisional Application Nos. 60/703,564; 60/703,571; 60/703,572; 60/703,643; 60/703,710; and 60/703,718 filed Jul. 25, 2005. Diagnostic agents provided by the present disclosure include entities that are inherently transporter substrates and that inherently have diagnostic activity. Diagnostic agents also include diagnostic conjugates. Diagnostic conjugates comprise a moiety that is a diagnostic agent and a substrate moiety that imparts transporter substrate activity to the diagnostic conjugate. Diagnostic agents and diagnostic conjugates that are transporter substrates may have a molecular weight less than about 1,500 Da and in certain embodiments, less than about 1,000 Da. Diagnostic agents and diagnostic conjugates that are transporter substrates may have a Vₘₐₓ that is at least about 5%, in certain embodiments, at least about 10%, in certain embodiments at least about 20%, in certain embodiments at least about 50%, and in certain embodiments, at least about 75%, and in certain embodiments, greater than about 100% of the Vₘₐₓ of a known substrate for the particular transporter. Examples of diagnostic agents include those that are capable of producing an image, for example, by optical imaging methods such as bioluminescence and fluorescence, magnetic resonance imaging (MRI), positron emission tomography (PET), single photon emission computed tomography (SPECT), radionuclide imaging, and computed tomography (CT). Examples of CT imaging and contrast agents include iobitridol, iopentol, ioversol and trypanoate sodium. Examples of optical imaging and contrast agents include A15, AO1987, CLIO-Cy5.5, Cy5.5-Annexin V, Cyp-GRD, Cy5.5 Endostatin, GPI-78, IR-786, NIR2-Folate, Pamidornate-IRDye98, QD-PSMA Ab J591, QD705-RGD, Cy5.5-R4-SC-CLIO, RGD-Dy5.5, [¹¹C]SIDAG, and Cy-5.5-trastuzumab. Examples of SPECT imaging and contrast agents include [¹²³I]VEGF165, ¹¹¹In-humAb4D5-8 Fab, ¹¹¹In-humAb4D5-8, RP748, [^{99m}Tc]MIBI, ¹¹¹In-DTPA-OC, Radioiodine-NP-4 F9ab′)2, [¹²³I]IL-2, ^{99m}Tc-HYNIC-EGF, ^{99m}Tc-HYNIC-annexin V, ^{99m}Tc-EC-endostatin, ^{99m}Tc-EC-folate, ^{99m}Tc-EC-C225, ¹¹¹In-DTPA-folate, ¹¹¹In-CYT-356, ^{99m}Tc-IMMU-4, radiolabeled anti-CD105 Mab, [¹²³I]FP-CIT, [¹²⁴I]FIAU, [^{123/125}I]IMPY, ¹¹¹In-2B8 Mab, [¹²³I]IMZ, ¹²³I-NP-4 Mab, and ¹³¹I-B1 Mab. Examples of MRS imaging and contrast agents include 6-fluoropyridoxol. Examples of MRI imaging and contrast agents include CLIO-Cy5.5, gadobenate, gadobutrol, gadoversetamide, and gadoteridol. Examples of PET imaging agents include N-[¹¹C]methylpiperidin-4-yl acetate, 1-[methyl-¹¹C]methionine, 7-[¹¹C]methoxy-1-methyl-9H-[3,4-b]indole, 3′deoxy-3′[¹⁸F]fluorothymidine, 6-[¹⁸F]fluorodopamine)-(3-[¹⁸F]fluoropropyl)-1-tyrosine, [¹⁸F]FMTP, [¹⁸F]fluoromisonidazole, 1-2,4-dihydroxy-6-[¹⁸F]fluorophenylalanine, and [¹⁸F]fluoro-2-deoxy-2-D-glucose. These examples of diagnostic agents have generally been developed to target particular tissue and diseases. Diagnostic moieties provided by the present disclosure include small molecules having imaging or contrast capability such as having a luminescent moiety, paramagnetic moiety, or a radioactive label and that do not necessarily have tissue or disease targeting capability. Such selective targeting capability is provided when a diagnostic moiety is linked to a transporter substrate moiety to form a diagnostic conjugate. A diagnostic moiety may be of sufficiently low molecular weight and/or have electrostatic properties such that when linked to a transporter substrate moiety, the resulting conjugate is a transporter substrate. For example, in certain embodiments, a diagnostic moiety, when linked to a transporter substrate moiety, will not render the resulting diagnostic conjugate much more hydrophilic, e.g., will not decrease the clogD by more than −2.

A diagnostic agent may be a substrate for any of the transporters overexpressed, for example in cancers such as GLUT1, GLUT3, GLUT5, LAT1, ENT1, MCT1, MCT4, and SMVT; or transporters expressed in the BBB such as GLUT1, LAT1, MCT1, TAUT1, OAT3, OATPB, GAT2, OCT3, OCTN2, SVCT2, CAT1, and BGT1. In certain embodiments, diagnostic agents may be a substrate for GLUT1, GLUT2, or GLUT3, in certain embodiments, for MCT1 or MCT4, and in certain embodiments, for LAT1, ENT1, or SMVT In certain embodiments, diagnostic agents may be a substrate for a transporter expressed in the BBB such as GLUT1, LAT1, MCT1, or OATPB. In certain embodiments, diagnostic agents may be a substrate for a transporter expressed the BBB such as TAUT1, GAT2, CAT1, or BGT1.

Therapeutic agents provided by the present disclosure include entities that are inherently transporter substrates and that inherently have therapeutic activity. Therapeutic agents also include therapeutic conjugates. Therapeutic conjugates comprise a moiety that is a therapeutic agent and a substrate moiety that imparts transporter substrate activity to a therapeutic conjugate. Therapeutic agents and therapeutic conjugates that are transporter substrates may have a molecular weight less than about 1,500 Da and in certain embodiments, less than about 1,000 Da. Therapeutic agents and therapeutic conjugates that are transporter substrates may have a Vₘₐₓ that is at least about 5%, in certain embodiments, at least about 10%, in certain embodiments at least about 20%, in certain embodiments at least about 50%, and in certain embodiments, at least about 75%, and in certain embodiments, greater than about 100% of the Vₘₐₓ of a known substrate for the particular transporter. In certain embodiments, the therapeutic moiety can be an antineoplastic agent. Examples of antineoplastic agents include, but are not limited to, melphalan, bromopyruvate, streptozoticoin, chlorozotocin, ranimustine, chlorambucil, cisplatin, acivicin, oxaliplatin, glufosfamide, gemcitabine, 5-fluorouridine, methotrexate, cladribine, mitobronitol, bendamustine, temozolomide, mitozolomide, irofulven, mitomycin C, doxifluridine, tegafur, and bis(chloroethyl)uracil. Other examples of chemotherapeutic agents useful in conjugates provided by the present disclosure include, but are not limited to, alemtuzumab, alitretinoin, allopurinol, altretamine, amifostine, anastrozole, arsenic trioxide, asparaginase, BCG, bexarotene, bicualutamide, bleomycin, buserelin, busulfan, calusterone, camptothecin, capecitabine, carboplatin, carmustine, celecoxib, chlorambucil, cisplatin, cladribine, cyclophosphamide, cytarabine, dacarbazine, dactinomycin, darbepoetin alfa, daunorubicin, denileukin diftitox, desleukin, dexamethasone, dexrazoxane, docetaxel, doxorubicin, dromostanolone propionate, epirubicin, estramustine, etoposide phosphate, etoposide, exemestane, filgrastim, floxuridine, fludarabine, fluorouracil, fluoxymestrone, flutamide, fludarabine, fulvestrant, gemcitabine, gemtuzumab ozogamicin, glutathione, goserelin acetate, hexamethylmelamine, hydroxyurea, ibritumomab tiuxetan, idarubicin, ifosfamide, imatinib mesylate, interferon alfa-2a, interferon alfa-2b, irinotecan, lenograstim, letrozole, leucovorin, leuprolide, levamisole, lomustine, mechlorethamine, megestrol acetate, melphalan, mercaptopurine, mesna, methotrexate, methoxsalen, methylprednisolone, mitomycin C, mitotane, mitoxantrone, nandrolone phenpropionate, nilutamide, nofetumomab, oprelvekin, oxaliplatin, paclitaxel, pamidronate, pegademase, pegaspargase, peglilgrastim, pentostatin, pipobroman, pirarubicin, plicamycin, porfimer sodium, prednisone, procarbazine, quinacrine, rasburicase, rituximab, sargramostim, sodium phenylbutyrate, streptozocin, tamoxifen, temozolomide, teniposide, testolactone, thioquanidine, thiotepa, topotecan, toremifene, tositumomab, trastuzumab, tretinoin, triptorelin, uracil mustard, valrubicin, vinblastine, vincristine, vinorelbine, and zoledronate.

Therapeutic agents may be substrates for any of the transporters expressed in cancers such as GLUT1, GLUT3, GLUT5, LAT1, ENT1, MCT1, MCT4, and SMVT; or transporters expressed in the BBB such as GLUT1, LAT1, MCT1, TAUT1, OAT3, OATPB, GAT2, OCT3, OCTN2, SVCT2, CAT1, and BGT1. In certain embodiments, therapeutic agents may be a substrate for GLUT1, GLUT2, or GLUT3, in certain embodiments, for MCT1 or MCT4, and in certain embodiments, for LAT1, ENT1, or SMVT In certain embodiments, therapeutic agents may be a substrate for a transporter substrate expressed in the BBB such as GLUT1, LAT1, MCT1, OATPB, GAT2, or OCT3. In certain embodiments, therapeutic agents may be a substrate for a transporter expressed in the BBB such as TAUT1, GAT2, CAT1, or BGT1.

In some embodiments, a diagnostic agent can also be a therapeutic agent. For example, many radioisotopes are suitable for imaging and also exhibit antineoplastic activity. In such cases, methods of diagnosis and methods of treatment can be combined.

Diagnostic and therapeutic conjugates can be prepared either by direct conjugation of a diagnostic or therapeutic moiety to a substrate moiety with a covalent bond, or by covalently coupling a di-functionalized linker precursor with a diagnostic or therapeutic moiety and a transporter substrate moiety. The linker precursor can be selected to contain at least one reactive functionality complementary to at least one reactive functionality of the diagnostic or therapeutic moiety and at least one reactive functionality of the transporter substrate moiety. Suitable complementary reactive groups are well known in the art and include, for example, those listed in Table 1.

TABLE 1
Complementary Binding Chemistries

| First Reactive Group | Second Reactive Group | Linkage |
|---|---|---|
| hydroxyl | carboxylic acid | ester |
| hydroxyl | haloformate | carbonate |
| thiol | carboxylic acid | thioester |
| thiol | haloformate | thiocarbonate |
| amine | carboxylic acid | amide |
| hydroxyl | isocyanate | carbamate |
| amine | haloformate | carbamate |
| amine | isocyanate | urea |
| carboxylic acid | carboxylic acid | anhydride |
| hydroxyl | phosphorous | phosphonate or |
| | | phosphate ester |

In certain embodiments, the covalent bond between a diagnostic or therapeutic moiety and a transporter substrate moiety is cleavable, and is capable of releasing the diagnostic or therapeutic agent or functional derivative thereof from the conjugate. The bond can be cleaved chemically and/or enzymatically. In certain embodiments, one or more enzymes present within a target cell, such as a diseased cell, may enzymatically cleave the diagnostic or therapeutic moiety of the conjugate, and in certain embodiments, one or more enzymes present in the stomach, intestinal lumen, intestinal tissue, blood, liver, brain, or any other suitable tissue of a mammal can enzymatically cleave the diagnostic or therapeutic moiety of the conjugate. The covalent bond may be cleaved after the conjugate has been translocated across the plasma membrane of a cell via a transporter. The covalent bond may be cleaved intracellularly, for example, within the cytoplasm of a target cell to intracellularly release the diagnostic or therapeutic moiety or functional derivative thereof. In certain embodiments, the covalent bond c may be cleaved after the conjugate has been translocated through a mediating cell or tissue to release the diagnostic or therapeutic moiety or functional derivative thereof in an extracellular fluid or within a target cell in which the conjugate is taken up by passive and/or active translocation mechanisms. For example, the covalent bond may be cleaved in the systemic circulation including the blood plasma of a patient following absorption of the conjugate through the intestinal epithelium via an active transporter, the covalent bond may be cleaved in the brain blood following passage of the conjugate through the BBB via an active transporter, or the covalent bond may be cleaved in the CNS after passage of the conjugate through the BBB via an active transporter.

Diagnostic agents and therapeutic agents provided by the present disclosure can be provided as panels comprising a plurality of diagnostic and/or therapeutic agents. Diagnostic and/or therapeutic agents included in panels exhibit characterized transporter substrate activity and utility as a diagnostic and/or therapeutic agent for certain diseases, disorders, or conditions. A clinician may select a diagnostic agent that is a substrate for a particular transporter from a panel of diagnostic agents to determine a level of transporter expression in a patient's tissue. In certain embodiments, a clinician may select a therapeutic agent that is a substrate for a particular transporter from a panel of therapeutic agents to treat a disease in a patient in which cells associated with the disease express the transporter.

Panels of diagnostic agents provided by the present disclosure may comprise a plurality of diagnostic agents. Each of the plurality of diagnostic agents may be a substrate for the same transporter or for one or more different transporters. Diagnostic agents may have inherent diagnostic activity or may be diagnostic conjugates. Diagnostic conjugates in a panel may comprise a diagnostic moiety bonded to a substrate moiety in which each diagnostic moiety and each substrate moiety can be the same or different. For example, for diagnosing certain cancers, a panel of diagnostic agents may include diagnostic agents that are substrates for one or more of the transporters GLUT1, GLUT3, GLUT5, LAT1, ENT1, MCT1, MCT4, and SMVT. Diagnostic agents may be diagnostic conjugates in which each diagnostic conjugate comprises the same diagnostic moiety coupled to a different substrate moiety, wherein the substrate moieties are selected from substrates for the cancer transporters GLUT1, GLUT3, GLUT5, LAT1, ENT1, MCT1, MCT4, and SMVT; and the BBB transporters GLUT1, LAT1, MCT1, TAUT1, OAT3, OATPB, GAT2, OCT3, OCTN2, SVCT2, CAT1, and BGT1. In certain embodiments, diagnostic conjugates may be a substrate for GLUT1, GLUT2, or GLUT3, in certain embodiments, for MCT1 or MCT4, and in certain embodiments, for LAT1, ENT1, or SMVT In certain embodiments, diagnostic conjugates may be a substrate for a transporter expressed in the BBB such as GLUT1, LAT1, MCT1, or OATPB. In certain embodiments, diagnostic conjugates may be a substrate for a transporter expressed the BBB such as TAUT1, GAT2, CAT1, or BGT1Panel of therapeutic agents provided by the present disclosure may comprise a plurality of therapeutic agents. Each of the plurality of therapeutic agents may be a substrate for the same transporter or for one or more different transporters. Therapeutic agents may have inherent therapeutic activity or can be therapeutic conjugates. Therapeutic conjugates in a panel may comprise a therapeutic moiety coupled to a substrate moiety in which each therapeutic moiety and each substrate moiety may be the same or different. For example, for treating certain cancers, a panel of therapeutic agents may include therapeutic agents that are substrates for one or more of the cancer transporters GLUT1, GLUT3, GLUT5, LAT1, ENT1, MCT1, MCT4, and SMVT; and/or one or more of the BBB transporters GLUT1, LAT1, MCT1, TAUT1, OAT3, OATPB, GAT2, OCT3, OCTN2, SVCT2, CAT1, and BGT1. Therapeutic agents may be therapeutic conjugates in which each therapeutic conjugate comprises the same therapeutic moiety coupled to a different substrate moiety, wherein the substrate moieties are selected from substrates for the cancer transporters GLUT1, GLUT3, GLUT5, LAT1, ENT1, MCT1, MCT4, and SMVT; and/or the BBB transporters GLUT1, LAT1, MCT1, TAUT1, OAT3, OATPB, GAT2, OCT3, OCTN2, SVCT2, CAT1, and BGT1. In certain embodiments, therapeutic conjugates may be a substrate for GLUT1, GLUT2, or GLUT3, in certain embodiments, for MCT1 or MCT4, and in certain embodiments, for LAT1, ENT1, or SMVT In certain embodiments, therapeutic conjugates may be a substrate for a transporter substrate expressed in the BBB such as GLUT1, LAT1, MCT1, or OATPB, GAT2, OCT3. In certain embodiments, therapeutic conjugates may be a substrate for a transporter expressed in the BBB such as TAUT1, GAT2, CAT1, or BGT1.

Panels provided by the present disclosure include pharmaceutical compositions comprising a diagnostic and/or therapeutic agent that are transporter substrates.

In certain embodiments, pharmaceutical compositions provided by the present disclosure comprise one or more diagnostic agents or therapeutic agents that are transporter substrates, preferably in purified form, together with a suitable amount of one or more pharmaceutically acceptable vehicles, so as to provide a form for proper administration to a patient. In certain embodiments, pharmaceutical compositions comprise a diagnostically effective amount of a diagnostic agent or a therapeutically effective amount of a therapeutic agent. In certain embodiments, pharmaceutical compositions comprise a diagnostically effective amount of a diagnostic agent and a therapeutically effective amount of a therapeutic agent.

One or more diagnostic agents or therapeutic agents provided by the present disclosure may be combined with pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, phosphate buffered saline (PBS), Ringer's solution, dextrose solution, and Hank's solution. In addition, the pharmaceutical composition or formulation can also include other carriers, adjuvants, or non-toxic, non-therapeutic, nonimmunogenic stabilizers, excipients, and the like. The compositions can also include additional substances to approximate physiological conditions, such as pH adjusting and buffering agents, toxicity adjusting agents, wetting agents, detergents, and the like (see, e.g., Remington's Pharmaceutical Sciences, Mace Publishing Company, Philadelphia, Pa., 21^{st} ed. (2005); for a brief review of methods for drug delivery, see, Langer, Science 1990, 249, 1527-1533).

Pharmaceutical compositions may be manufactured, for example, by means of conventional mixing, dissolving, granulating, dragee-making, levigating, emulsifying, encapsulating, entrapping, and lyophilizing processes. Pharmaceutical compositions can be formulated in a conventional manner using one or more physiologically acceptable carriers, diluents, excipients, or auxiliaries that facilitate processing of compounds disclosed herein into preparations that may be used pharmaceutically. Appropriate formulation may be dependent upon the route of administration chosen.

Pharmaceutical compositions of the present disclosure can be administered to a patient by any appropriate method including, for example, topically, orally, intranasally, intradermally, subcutaneously, intrathecally, intramuscularly, topically, intravenously, or injected directly to a site of the disease such as to cancerous tissue. For parenteral administration, pharmaceutical compositions of the present disclosure can be administered as injectable dosages of a solution or suspension of a diagnostic or therapeutic agent in a physiologically acceptable diluent with a pharmaceutically acceptable vehicle that can be a sterile liquid such as water, oils, saline, glycerol, or ethanol. Additionally, auxiliary substances, such as wetting or emulsifying agents, surfactants, pH buffering substances, and the like may be present in compositions. Other components of pharmaceutical compositions include those of petroleum, animal, vegetable, or synthetic origin, for example, peanut oil, soybean oil, and mineral oil. In general, glycols such as propylene glycol or polyethylene glycol are useful liquid carriers, particularly for injectable solutions.

Pharmaceutical compositions provided by the present disclosure may be prepared as injectables, either as liquid solutions or suspensions. Solid forms of pharmaceutical compositions suitable for solution in, or suspension in, liquid vehicles prior to injection may also be prepared. Pharmaceutical compositions may also be emulsified or encapsulated in liposomes or microparticles such as polylactide, polyglycolide, or copolymers thereof for enhanced adjuvant effect (see Langer, Science 1990, 249, 1527; Hanes, Advanced Drug Delivery Reviews 1997, 28, 97-119). Pharmaceutical compositions may be administered in the form of a depot injection or implant preparation that may be formulated in such a manner as to permit a sustained or pulsatile release of the active ingredient.

Pharmaceutical compositions for oral administration may be in the form of, for example, tablets, pills, powders, lozenges, sachets, cachets, elixirs, suspensions, emulsions, solutions, or syrups. Examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methylcellulose. Preserving agents such as methyl- and propylhydroxy-benzoates; sweetening agents; and flavoring agents may also be included. Depending on the formulation, compositions may provide rapid, sustained or delayed release of the active ingredient after administration to a patient. Polymeric materials may be used for oral sustained release delivery (see “Medical Applications of Controlled Release,” Langer and Wise (eds.), CRC Pres., Boca Raton, Fla. (1974); “Controlled Drug Bioavailability,” Drug Product Design and Performance, Smolen and Ball (eds.), Wiley, New York (1984); Ranger and Peppas, J. Macromol. Sci. Rev. Macromol Chem. 1983, 23, 61; see also Levy et al., Science 1985, 228, 190; During et al., Ann. Neurol. 1989, 25, 351; Howard et al., J. Neurosurg, 1989, 71, 105). Sustained release may be achieved by encapsulating conjugates within a capsule, or within slow-dissolving polymers. Examples of useful slow-dissolving polymers include sodium carboxymethylcellulose, hydroxypropylcellulose, hydroxypropylmethylcellulose, and hydroxyethylcellulose. Other useful cellulose ethers have been described (Alderman, Int. J. Pharm. Tech. & Prod. Mfr. 1984, 5(3), 1-9) and factors affecting drug release have been described in the art (Bamba et al., Int. J. Pharm. 1979, 2, 307).

When a diagnostic or therapeutic agent provided by the present disclosure is acidic, the agent may be included in any of the above-described formulations as the free acid, a pharmaceutically acceptable salt, a solvate, hydrate, or N-oxide thereof. Pharmaceutically acceptable salts can substantially retain the activity of the free acid, may be prepared by reaction with bases, and may be more soluble in aqueous and other protic solvents than the corresponding free acid form. In some embodiments, sodium salts of a diagnostic or therapeutic agent provided by the present disclosure may be used in the above described formulations.

The components of pharmaceutical compositions are preferably of high purity and are substantially free of potentially harmful contaminants (e.g., at least National Food (NF) grade, generally at least analytical grade, and more typically at least pharmaceutical grade).

To the extent that a given compound must be synthesized prior to use, the resulting product may be substantially free of any potentially toxic agents, particularly any endotoxins that may be present during the synthesis or purification process. Pharmaceutical compositions are usually made under GMP conditions. Pharmaceutical compositions for parenteral administration are usually sterile and substantially isotonic.

Effective dosage amounts and regimes (amount, frequency, and duration of administration) of pharmaceutical compositions comprising a therapeutic agent provided by the present disclosure may be determined according to any one of several well-established protocols. For example, animal studies (e.g., mice, rats) are commonly used to determine the maximal tolerable dose of the bioactive agent per kilogram of weight. In general, at least one of the animal species tested is mammalian. The results from animal studies may be extrapolated to determine doses for use in other species, such as humans.

The amount of diagnostic or therapeutic agents that will be effective in the diagnosis or treatment of a particular disease may depend on the nature of the disease, and can be determined by standard clinical techniques known in the art. In vitro and/or in vivo assays may be used to identify optimal dosage ranges. The amount of a diagnostic or therapeutic agent administered to a patient in a particular dose or dosing regimen may depend on, among other factors, the patient being treated, the weight of the patient, the severity of the disease, the manner of administration, and the judgment of the prescribing physician.

Dosage forms may be adapted to be administered to a patient at any appropriate frequency. Dosing may be provided alone or in combination with other diagnostic or therapeutic agents and may continue as long as required for effective diagnosis or treatment. Appropriate dosage ranges may be determined by methods known to those skilled in the art.

Diagnostic agents provided by the present disclosure that are transporter substrates and pharmaceutical compositions thereof may be used for in vivo diagnostics. Diagnostic agents and diagnostic compositions may be administered to a patient and may be preferentially taken up by cells of a tissue expressing a transporter. Diagnostic agents that are transporter substrates may be taken up by any tissue in the patient's body that expresses the transporter, including diseased and non-diseased tissue. The imaging activity of the diagnostic agent may then be detected using an imaging method as appropriate for the diagnostic agent. Examples of diagnostic imaging techniques include positron emission tomography (PET), magnetic resonance imaging (MRI), computed tomography (CT), and single photon emission computer tomography (SPECT). Transporter-targeted diagnostic agents may provide information about, for example, the presence of a disease, the spatial characteristics of a disease, e.g., the presence and/or size of a disease, the distribution of a disease within an organ or the body of a patient, the type of disease, an absolute level of transporter expression in cells of a diseased tissue, and/or a relative level of transporter expression in cells of diseased and non-diseased tissue relative to cells of non-diseased or normal tissue from the same or different organ as the diseased tissue. In certain embodiments, diagnostic agents may be used to determine the expression profiles of one or more transporters. A transporter expression profile includes the absolute level of expression of a transporter and the relative level of expression of the transporter in one or more tissues in a patient. A transporter expression profile also includes the level of expression for one or more transporters expressed in cells of tissue from different organs, in cells of different tissue from the same organ, in cells of the same cell type from the same organ, and in cells of the same cell type from a different organ. The tissue used for comparison of a transporter expression profile may be from a single patient or from two or more patients and may represent a norm or model derived from a multiplicity of patients. Cells of a tissue used for determination of a relative transporter expression level may be from diseased tissue, tissue that is known to be free of the disease, or tissue that is known to be unaffected by the presence of the disease.

Upon determining the absolute and/or relative transporter expression level in cells of tissue of a patient, a clinician may use the determined transporter expression level to select a therapeutic agent for treating a disease in the patient. Treatment may be indicated when, for example, a determined transporter expression level in cells of a tissue is higher than a predetermined level, a differential transporter expression level between cells of a tissue and cells of other tissue of the same or different organ in the patient is above a predetermined level, or a differential transporter expression level between cells of a tissue and cells of other tissue in a patient's body is above a predetermined level. Furthermore, cells used for comparison with a predetermined level may be of the same cell type. Treatment may also be indicated when a determined transporter expression level is less than a predetermined level. In certain embodiments, a selected therapeutic agent can be a substrate for the same transporter as that of the diagnostic agent used to determine the expression level.

The response of a patient to treatment in which tissue affected by the disease expresses a transporter may be monitored using diagnostic agents of the present disclosure. A diagnostic agent that is a transporter substrate for a transporter expressed by cells of a diseased tissue in a patient may be administered to the patient before treatment, during treatment, and/or after treatment with a therapeutic compound. Treatment may be with a transporter-targeted therapeutic agent provided by the present disclosure or other therapeutic compound appropriate for treating the disease. In certain embodiments, higher differential transporter expression levels for cells of a tissue are useful for transporter-targeted therapy. Selection of a particular therapeutic agent may depend on other factors as will be appreciated by those skilled in the art. In certain embodiments, cells of a tissue expressing a transporter and imaged using a diagnostic agent may be cells capable of mediating delivery of a therapeutic agent to a target tissue. For example, brain capillary epithelial cells and intestinal epithelial cells express transporters that mediate translocation of nutrients from the blood or intestine to the CNS or systemic circulation, respectively. In such cases, transporter-targeted therapy may involve selecting and administering to a patient a therapeutic agent that is a substrate for a transporter expressed by the mediating cells. After translocation of the therapeutic agent into a mediating cell, the therapeutic agent may be translocated from the cell, or in the case of a therapeutic conjugate, the therapeutic moiety can be cleaved and the cleaved therapeutic moiety translocated from the mediating cell to exert a therapeutic effect on a target cell by any appropriate mechanism. For example, the translocated therapeutic agent or metabolite thereof may exert a therapeutic effect on the target tissue through extracellular mechanisms or intracellular mechanisms where the therapeutic agent or metabolite thereof is taken up by a target cell by passive and/or active transport. The therapeutic agent may be a substrate for the same transporter, a substrate for a different transporter expressed by the diseased tissue, or not be a transporter substrate. Following administration of the diagnostic agent to the patient, the diagnostic agent may be imaged. The intensity and/or spatial distribution of the image may be determined and compared to the intensity and/or spatial distribution of an image obtained at an earlier and/or later time during the treatment. In certain embodiments, transported diagnostic agents may provide a spatial measure of diseased tissue in a patient's body. A successful treatment may be indicated, for example, by a decrease in the size, volume, or distribution of the diseased tissue during and/or following treatment. In certain embodiments, successful treatment may also be indicated when there is no increase in the size of the diseased tissue or metastasis of the diseased tissue.

Use of a diagnostic agent that is a substrate for a transporter may facilitate the ability to image those tissues that are targeted by a therapeutic agent that is a substrate for the same transporter. Diagnostic agents may be used to monitor treatment of a disease in a patient in which disease is characterized by an atypical transporter expression level in cells of a tissue of the patient affected by the disease. Such methods of monitoring treatment include measuring a first transporter expression level in cells of the tissue, measuring a second transporter expression level in cells of the tissue at a time after the first expression level is measured and after a regimen of the treatment, and, in certain embodiments, comparing the second level with the first expression level. The first expression level may be measured before or after any treatment regimen has been initiated. For example, before treatment, a diagnostic agent, which in certain embodiments may be a diagnostic conjugate that is substrate for a transporter, can be administered to a patient and the size of diseased tissue such as cancerous tissue determined. A therapeutically active agent that is a substrate for the same transporter may then be administered to the patient. The response to the treatment may then be determined by administering the same diagnostic agent, determining the size of the diseased tissue and comparing to the size of the diseased tissue previously measured. Depending on the response, the treatment regimen may then be continued, modified, or discontinued. In certain embodiments, the second expression level may be compared with (i) the first expression level and at least one of: (ii)(a) an expression level of the transporter measured in cells of a second tissue of the patient that is known to be free of the disease; (ii)(b) an expression level of the transporter measured in cells of a second tissue of the patient that is known to be unaffected by the presence of the disease, (ii)(c) an expression level of the transporter measured in cells of a tissue of another patient who is free of the disease, or (ii)(d) an expression level of the transporter derived from a multiplicity of patients who are free of the disease. In certain embodiments, the first tissue and the second tissue are from the same organ, and in certain embodiments, the cells of the first tissue and the cells of the second tissue are of the same cell type. The cells of a diseased tissue may be characterized by an atypical expression level of a transporter, which may either higher or lower compared to the expression in normal tissue. The progress of a particular regimen of treatment may be followed by monitoring the change in the transporter expression level in cells of diseased and/or non-diseased tissue at time intervals before, during, and/or after any regimen of treatment. A successful regimen of treatment may be indicated by a change in the transporter expression level from atypical to normal transporter expression level. A regimen of treatment may include administering to a patient a therapeutic agent that is a substrate for the transporter being monitored.

Transporter-targeted diagnostic agents may also be used to monitor preventive treatment. In such methods, successful preventive treatment may be indicated by a failure to detect new tissue that differentially expresses, e.g., over-expresses or under-expresses, a transporter compared to the level of transporter expression in cells of non-diseased tissue.

For preventive use, therapeutic agents that are transporter substrates and pharmaceutical compositions thereof may be administered to a patient susceptible to, or otherwise at risk of, a disease in which tissue associated with the disease expresses an active transporter, such as for example, certain of the cancers disclosed herein, in an amount and frequency sufficient to eliminate or reduce the risk, lessen the severity, or delay the outset of the disease, including biochemical, histological, and/or behavioral symptoms of the disease, its complications and intermediate pathological phenotypes presenting during development of the disease. For certain diseases, use of transporter-targeted therapeutic agents may be used to prevent or minimize the spread of the disease. For example, a transporter targeted therapeutic agent may be used in the treatment of cancer to prevent or minimize tumor metastasis.

Delivering therapeutic agents preferentially to diseased tissue relative to non-diseased tissue may enhance the efficacy of treatment. Diseased tissue that expresses a particular transporter may be treated more effectively by targeting the diseased tissue with a therapeutic agent that is a substrate for the expressed transporter. Transporter expression in a tissue and/or tissues in a patient's body may be determined using in vitro and/or in vivo methods. Transporter expression may be determined using in vitro assay methods known in the art. For example, biopsy samples can be obtained from a patient and the level of transporter mRNA and/or transporter protein expression may be determined using, for example, Western blot, immunohistochemistry, Northern blot, Southern blot, PCR, DNA sequencing, or other methods known to those skilled in the art. Transporter expression may also be determined using in vivo methods comprising administering a diagnostic agent of the present disclosure or other in vivo diagnostic agent to a patient. Diagnostic agents that are transporter substrates may be administered to a patient having or suspected of having a disease. Diagnostic agents may be administered systemically or administered directly to a diseased tissue. Imaging the diagnostic agent following administration to a patient may provide a measure for the level of transporter expression in the diseased tissue and in non-diseased tissue. In certain embodiments, the diagnostic agent may provide an absolute level of transporter expression, and/or a relative level of transporter expression. The relative level of transporter expression may be determined with respect to normal tissue from the same organ as the diseased tissue, with respect to the transporter expression level in other tissue in the patient's body, and/or with respect to the expression level in tissue from other patients. The relative level of transporter expression may be determined in reference to one or more qualitative or quantitative metrics established, for example, by assessing transporter expression in tissue samples from multiple patients and the response to treatment regimens, and in particular to transporter-targeted treatment regimens. A diagnostic agent may also provide a measure of the disposition or spatial distribution of tissue or tissues expressing a transporter. This information may be used to select a therapeutic agent for treating a disease, select patients for treatment with a therapeutic agent that is a substrate for the transporter, and/or design a transporter-targeted treatment regimen.

In certain embodiments, it may be desirable to determine a differential transporter expression level between diseased and non-diseased tissue. A differential transporter expression level may be determined by comparing a transporter expression level in diseased tissue to a transporter expression level in non-diseased or normal tissue. Non-diseased tissue may include tissue from the same organ as the diseased tissue and/or tissue from other organs, fluids, etc. in the patient's body. A relative level of transporter expression may also include differential expression within a diseased tissue having differentiated cell types. For example, solid tumors may exhibit regions that are vascularized to various extents resulting in hypoxic regions in which sugar transporters are more highly expressed than cells in highly vascularized regions of the same tumor. The relative level of transporter expression may also be determined by comparing a level of transporter expression in diseased tissue to a norm or predetermined level. A norm or predetermined level may be derived from assessment of the transporter expression levels, for example, in non-diseased and/or diseased tissue from other patients, and may reflect the results of clinical trials, experience, and the judgment of the treating physician.

Patients having a disease exhibiting a greater differential transporter expression, e.g., cells of diseased tissue exhibit a higher level of transporter expression than non-diseased tissue, are expected to respond more favorably to treatment with a therapeutic agent that is a substrate for the transporter and to experience fewer adverse effects associated with the effects of the therapeutic agent on non-diseased tissue. It is also to be appreciated that patients having a disease exhibiting differential transporter expression may be successfully treated with comparatively lower amounts of a transporter-targeted therapeutic agent and thereby minimize the potential for adverse effects of the therapeutic agent on non-diseased tissue and/or of the patient's response to the administered transporter-targeted therapeutic agent.

Determination of a differential level of transporter expression may also be used to select therapeutic agents that preferentially target diseased tissue relative to surrounding tissue and/or tissue in other body organs. Certain therapeutic agents, such as for example, antineoplastic chemotherapeutic agents will not only affect cancer cells, but will also affect non-cancer cells. Determining a differential transporter expression level of a diseased tissue relative to non-diseased tissue may be used to select a therapeutic agent that is a substrate for transporters that are differentially expressed in the diseased tissue and that will therefore provide enhanced efficacy.

Determination of a differential transporter expression level may also be used to establish dosage levels and/or treatment regiments. For example, it may be appropriate to treat a disease known to overexpress a transporter relative to other tissue with a higher dosage of a therapeutic agent since adverse effects resulting from transport of the therapeutic agent into non-diseased or normal tissue is potentially reduced. The ability of the patient to tolerate higher doses of a transporter targeted therapeutic agent may result in more efficacious treatment. It will be appreciated that differential transporter expression may be used not only to select the amount of therapeutic agent administered in a single dosage, but also may be used to select a treatment regimen comprising the administration of multiple doses of one or more therapeutic agents over a period of time that will be efficacious in treating the disease.

In certain methods, a first step in transporter-targeted treatment may be to determine whether a disease in a patient expresses one or more transporters. Determining a level of transporter activity may be accomplished by administering a diagnostic agent that is a transporter substrate to the patient. In certain embodiments, more than one diagnostic agent may be administered to the patient with each diagnostic agent being a substrate for a different transporter. In this way, the transporter expression profile of the diseased tissue for more than one active transporter may be determined. After determining that cells of a tissue associated with the disease expresses a certain transporter, a therapeutic agent that is a substrate for the same transporter can be selected and administered to the patient to treat the disease. In certain embodiments, the therapeutic agent may be selected from a panel or plurality of therapeutic agents. A therapeutically effective amount of the selected therapeutic agent may then be administered to the patient.

In certain embodiments of methods of treating a disease in a patient, cells of a tissue in a patient associated with a disease may be screened to determine whether the tissue expresses a transporter at an expression level above a predetermined expression level. Cells of the tissue associated with the disease may be cells affected by the disease such as cancerous cells, or cells capable of mediating delivery of a therapeutic agent to cells and/or tissue affected by a disease such as BBB tissue. A predetermined transporter expression level may be a level of expression that indicates the presence of the disease. A predetermined level may be derived for example, by evaluating the expression of cells of non-disease and diseased tissue, as well as by other methods for establishing diagnosis criteria known to those skilled in the art. If, after screening, cells of the tissue are found to express the transporter above the predetermined level, then a therapeutic agent can be selected that is a substrate for the transporter. The therapeutic agent may be selected from a plurality of therapeutic agents, each of the plurality of therapeutic agents being therapeutically effective for treating the disease. Each of the plurality of therapeutic agents may be a substrate for a transporter, which can be the same transporter or different transporters. One or more of the plurality of therapeutic agents as well as the selected therapeutic agent may be a therapeutic conjugate comprising a therapeutic moiety and a substrate moiety. Finally, a therapeutically effective amount of the selected therapeutic agent may be administered to the patient for treating the disease. In certain embodiments, the tissue is cancerous tissue, such as breast cancer, lung cancer, colon cancer, and ovarian cancer, and in certain embodiments, the cancerous tissue comprises a tumor. In certain embodiments of methods of treating a disease, the tissue is cancerous tissue, the transporter is selected from GLUT1, GLUT3, GLUT5, LAT1, ENT1, MCT1, MCT4, and SMVT, and the therapeutic agent may comprise an antineoplastic agent. In certain embodiments of methods of treating a disease, the tissue comprises brain or CNS tissue, and the therapeutic agent is also a substrate for a transporter expressed in blood-brain-barrier tissue. Such methods of treating a disease in a patient can be useful when tissue associated with the disease has a variable transporter expression profile across a population of patients having the disease, and thereby can be useful for tailoring transporter-directed therapy to individual patients. In certain embodiments of methods of treating a disease, the cells of the tissue associated with the disease may express different transporters across the patient population, and in certain embodiments, the cells of the tissue associated with the disease may express different levels of the same transporter across the patient population. Some patients within the patient population may have cells of the tissue associated with a disease that have the same transporter expression profile, that may express the same transporters, and/or that may express certain transporters at the same level as cells associated with tissue of the disease in other patients. Nevertheless, methods of treating a disease of the present disclosure are particularly useful for identifying patients and/or selecting therapeutic agents for transporter-targeted therapy in which cells associated with the disease have a variable transporter expression profile across a population of patients having the disease.

In certain embodiments of methods of treating a disease, screening may comprise administering a diagnostic agent that is a substrate for the transporter to the patient, measuring the uptake of the diagnostic agent into the cells of the tissue associated with the disease such as cancerous tissue, and evaluating the measured uptake of the diagnostic agent to determine whether the cells of the tissue express at least the predetermined expression level of the transporter. In such embodiments, the diagnostic agent may be capable of producing an image and measuring the uptake of the diagnostic agent may include recording an image of the cells of the tissue after administering the diagnostic agent to the patient. Recording an image may include any of the methods for in vivo imaging known to those skilled in the art including those disclosed herein.

In certain embodiments of methods of treating a disease, screening may comprise administering a diagnostic agent that is a substrate for the transporter to the patient, measuring transport of the diagnostic agent through the cells of the tissue associated with the disease such as blood-brain barrier tissue, and evaluating the measured transport of the diagnostic agent to determine whether the cells of the tissue express at least the predetermined expression level of the transporter. In such embodiments, the diagnostic agent may be capable of producing an image and measuring the transport of the diagnostic agent can include recording an image of cells that the diagnostic agent is transported into after the transport through the cells of the tissue and after administering the diagnostic agent to the patient.

Methods provided by the present disclosure include methods of determining an expression level of a transporter in cells of a tissue associated with a disease such as cancerous tissue in a patient. Such methods may comprise administering a diagnostic conjugate that is a substrate for the transporter to the patient, measuring the uptake of the diagnostic conjugate into cells of the tissue, and evaluating the measured uptake of the diagnostic conjugate to determine the expression level of the transporter in the cells of the tissue. The cells of the tissue associated with the disease may have a variable transporter expression profile across a population of patients having the disease. In such methods, the diagnostic conjugate may be capable of producing an image and the measuring of the uptake of the diagnostic conjugate may comprise recording an image of the cells of the tissue that take up the diagnostic conjugate after administering the diagnostic conjugate to the patient.

Methods provided by the present disclosure include methods of determining an expression level of a transporter in cells of a first tissue associated with a disease in a patient such as tissue capable of mediating the delivery of a therapeutic agent that is a substrate for the transporter expressed by the mediating tissue to a tissue affected by the disease. In certain embodiments, a first tissue comprises blood-brain barrier tissue. Such methods may comprise administering a diagnostic agent that is a substrate for the transporter to the patient, measuring the transport of the diagnostic agent through cells of the first tissue, and evaluating the measured transport of the diagnostic agent to determine the expression level of the transporter in the cells of the first tissue after administering the diagnostic agent to the patient. In such methods, the diagnostic agent may be capable of producing an image and the measuring of the transport of the diagnostic agent may comprise recording an image of cells of a second tissue that the diagnostic agent is transported into after the transport through the cells of the first tissue and after administering the diagnostic agent to the patient.

In certain embodiments of methods of treating a disease in a patient, methods of determining an expression level of a transporter in a tissue affected by a disease in a patient, and methods of determining an expression level of a transporter in a first tissue associated with a first tissue associated with a disease in a patient, a diagnostic agent may be a diagnostic conjugate and/or a therapeutic agent can be a therapeutic conjugate.

In certain embodiments, diagnostic agents that are transporter substrates may be used to determine whether a disease in a patient is suitable to be treated with a therapeutic agent that is a known substrate for a transporter. Certain therapeutic agents that are transporter substrates may be effective for treating a disease. However, tissue associated with a disease may not always express the same transporter(s) in all patients, or may not express a particular transporter to the same extent, either in an absolute or relative, e.g., relative to non-diseased tissue, sense. For example, cells of a tissue associated with a disease may exhibit a variable transporter expression profile across a population of patients having the disease. Patients having a disease wherein cells of tissue of the patient associated with the disease express a transporter and who would be favorably disposed to being treated with a therapeutic agent that is a substrate for the same transporter can be identified by determining the level of transporter expression in cells of tissue associated with the disease. Those patients in which cells of tissue associated with a disease express a certain transporter or in which expression of the transporter in cells of tissue associated with a disease is above a predetermined level may be candidates for treatment with a therapeutic agent that is a substrate for the same transporter. In certain embodiments, methods of determining whether a disease in a patient is suitable to be treated with a therapeutic agent that is a known substrate for a transporter comprises measuring an expression level of the transporter in cells of a tissue of the patient known to be associated with the disease, comprising the measured expression level of the transporter with a minimum predetermined expression level known to be useful for the therapeutic agent to be effective in treating the disease, and determining that the disease is suitable to be treated with the therapeutic agent if the measured expression level is at least the minimum predetermined expression level. In certain embodiments, cells of a tissue associated with the disease may have a variable transporter expression profile across a population of patients having the disease. In certain embodiments, the tissue can be selected from cancerous tissue and in certain embodiments from brain and CNS tissue. In embodiments wherein the tissue is cancerous tissue and the therapeutic agent may comprise an antineoplastic agent. In embodiments wherein the tissue is selected from brain and CNS tissue, the therapeutic agent may be a substrate for a transporter expressed in blood-brain-barrier tissue. In certain embodiments of methods of determining whether a disease in a patient is suitable to be treated with a therapeutic agent that is a known substrate for a transporter, the therapeutic agent may be a therapeutic conjugate. Measuring an expression level of the transporter in cells of a tissue of the patient known to be associated with the disease may be by any method known to those skilled in the art, including those methods disclosed herein.

Methods provided by the present disclosure also include methods of determining the presence of a disease in a patient in which the disease is characterized by an atypical level of expression of one or more transporters in cells of a tissue of the patient associated with the disease. Such methods may be useful when the presence of disease in a tissue affected by the disease has been correlated with or is believed to be correlated with transporter expression levels in diseased and non-diseased tissue. For example, an expression level of a transporter expressed by a tissue that is atypical, e.g., does not correspond to the expression level of the transporter, may indicate the presence of a disease in the tissue. An atypical expression level can be higher than a normal expression level or less than a normal transporter expression level for the tissue. A normal transporter expression level may be determined from a tissue from the same or different patient known to be free of the disease, from a tissue from the same or different patient known to be unaffected by the presence of the disease, or derived from a multiplicity of patients who are free of the disease. Such methods may comprise measuring a first expression level of a transporter in cells of a first tissue of a patient and comparing the first expression level to a second expression level of the transporter. In certain embodiments, the second expression level of the transporter may be: (i) an expression level of the transporter measured in cells of a second tissue of the patient that is known to be free of the disease; (ii) an expression level of the transported measured in cells of a second tissue of the patient that is known to be unaffected by the presence of the disease; (iii) an expression level of the transporter measured in cells of a tissue of another patient who is free of the disease; or (iv) an expression level derived from a multiplicity of patients who are free of the disease. The first tissue, the second tissue, and the tissue of another patient may be of the same organ. The cells of the first tissue, the cells of the second tissue, and the cells of a tissue from another patient may be of the same cell type. In certain embodiments, the expression level of the transporter in the first tissue is atypical if the first and second transporter expression levels differ by more than a predetermined value. An atypical expression level of the transporter in the first tissue may indicate the presence of a disease in the first tissue. The first and/or second expression levels of the transporter may be determined by methods known to those skilled in the art including those disclosed herein, for example, by methods comprising administering a diagnostic agent, including a diagnostic conjugate that is a substrate for the transporter and to a patient. If the presence of a disease in a patient is established, a therapeutically effective amount of a therapeutic agent that is a substrate for the same transporter as used to determine the presence of the disease may be administered to the patient to treat the disease. In certain embodiments, the tissue affected by the disease is cancerous tissue, including tumors, and the therapeutic agent may be an antineoplastic agent.

In certain embodiments, therapeutic agents that are transporter substrates may be available for treating a disease in a patient. For those therapeutic agents that are actively transported into a cell and that exert their therapeutic effect intracellularly, the efficacy of the therapeutic agent may depend on the level of transporter expression in the diseased tissue. A high transporter expression level in diseased tissue and/or a higher transporter expression level in diseased tissue relative to non-diseased or normal tissue may provide a motivation for undertaking transporter directed therapy. Diagnostic agents that are substrates for the same transporter as expressed in a diseased tissue may be used to determine whether a patient is more likely to be successfully treated with transporter-targeted therapeutic agents. For patients having the same or similar disease, the absolute or relative expression level of a particular transporter by the pathological tissue may vary for each patient. Thus, where efficacy depends upon transporter mediated uptake of a therapeutic agent, patients can respond differently to the same or similar treatment. For example, for patients having a disease that exhibits a low level of transporter expression, a particular transporter targeted therapy may not be effective or as effective, and for patients in which the disease exhibits a low level of differential expression, a transporter-mediated therapy may produce unacceptable side effects. Using transporter-targeted diagnostic agents and methods disclosed herein, patients having a disease in which a transporter is expressed can be identified, and selected for transporter targeted treatment based on the absolute or relative transporter expression level in the diseased tissue.

Therapeutic agents provided by the present disclosure that are transporter substrates and pharmaceutical compositions thereof can be used in methods of treating a patient having a disease, such as cancer. Examples of tumors amenable to treatment include cancers of the bladder, brain, breast, colon, esophagus, kidney, leukemia, liver, lung, oral cavity, ovary, pancreas, prostate, skin, stomach, and uterus. Therapeutic agents that are transporter substrates and compositions thereof may be useful for treating solid tumors, such as sarcomas, lymphomas, and carcinomas. Examples of cancers for treatment are those disclosed in Table 3 in which expression of an active transporter is higher in the cancer than in normal cells from the same tissue. Examples of these cancers include brain cancers, such as astrocytoma, glioblastoma multiforme, malignant ependymana, and medullablastoma. Examples of breast cancers amenable to treatment with transporter-targeted therapeutic agents include infiltrating ductal adenocarcinoma, ductal adenocarcinoma, and lobular adenocarcinoma. Examples of lung cancers amenable to treatment with transporter-targeted therapeutic agents include squamous cell carcinoma and epidermoid carcinoma. Examples of colon cancers amenable to treatment with transporter-targeted therapeutic agents include colon adenocarcinoma, medullary carcinoma, and mucinous carcinoma. Examples of prostate cancers amenable to treatment with transporter-targeted therapeutic agents include prostate sarcoma. Other diseases for which diagnostic agents, therapeutic agents, and methods of using such diagnostic and therapeutic agents are useful include diseases characterized by tissue affected by the disease or associated with the disease that overexpresses a transporter and/or that exhibits a variable transporter expression profile across a patient population. Examples of such diseases include cancer, prostate hyperplasia, certain heart diseases such as cardiomyopathy, and certain liver diseases such as sclerosis.

When used to treat a disease, a therapeutically effective amount of one or more therapeutic agents provided by the present disclosure may be administered singly or in combination with other agents including other pharmacologically active agents. For example, in the treatment of a patient suffering from cancer, a dosage form comprising a therapeutic agent provided by the present disclosure may be administered in conjunction with an anti-cancer agent such as desleukin, adriamycin, aldesleukin, alemtuzumab, alitretinoin, allopurinol, altretamine, amifostine, anastrozole, arsenic trioxide, asparaginase, bevacizumab, bexarotene, bicalutamide, betamethasone, bleomycin, busulfan, calusterone, camptothecin, capecitabine, carboplatin, carmustine, celecoxib, chlorambucil, cisplatin, cladribine, cyclophosphamide, cytarabine, dacarbazine, dactinomycin, darbepoetin alfa, daunorubicin, denileukin diflitox, dexrazoxane, diethylstilbestrol, docetaxel, doxorubicin, dromostanolone propionate, dronabinol, epirubicin, epoetin alpha, erlotinib, estramustine, etidronate, etoposide phosphate, etoposide, exemestane, filgrastim, floxuridine, fluconazole, fludarabine, fluorouracil, fulvestrant, gemcitabine, gemtuzumab ozogamicin, goserelin acetate, granisetron, hexamethylmelamine, hydroxyurea, ibritumomab tiuxetan, idamycin, idarubicin, ifosfamide, imatinib mesylate, interferon alfa-2b, irinotecan, letrozole, leucovorin, levamisole, lomustine, meclorethamine, megestrol acetate, melphalan, mercaptopurine, mesna, methotrexate, methoxsalen, mitomycin C, mitotane, mitoxantrone, nandrolone phenpropionate, nofetumomab, octreotide, ondansetron, oprelvekin, oxaliplatin, paclitaxel, pamidronate, pegademase, pegaspargase, peglilgrastim, pentostatin, pilocarpine, pipobroman, plicamycin, porfimer sodium, procarbazine, quinacrine, rasburicase, rituximab, sargramostim, sqalamine, streptozocin, tamoxifen, temozolomide, teniposide, testolactone, thioquanidine, thiotepa, Tice BCG, TNP 470, topotecan, toremifene, tositumomab, trastuzumab, tretinoin, uracil mustard, valrubicin, vinblastine, vincristine, vinorelbine, zoledronate, or combinations of any of the foregoing.

Administration of pharmaceutical compositions of the present disclosure can be combined with administration of other therapeutic and pharmacologically active agents. The additional agents are not necessarily substrates for the same transporter as the therapeutic agent of the composition, and in certain embodiments are not substrates for any transporter. The additional agents may increase the efficacy of the therapeutic agent by, for example, increasing the bioavailability of the therapeutic agent and/or augment the activity of the therapeutic agent. For example, the additional agent may be, for example, a chemotherapeutic sensitizing agent that inherently renders a cell susceptible to radiation damage or is linked to an antineoplastic component that renders a cell susceptible to radiation damage.

Incorporation of radioisotopes such asboron (¹⁰B) into transporter-targeted therapeutic agents can allow boron neutron capture therapies (BNCT) in which low-energy neutron irradiation is used to induce radioactive decay and release of higher energy particles that are toxic to cells (see e.g., Barth et al., Clin Cancer Res 2005, 11(11), 3987-4002). One advantage of a combined radiotherapy approach is that radioactive therapeutic agents that preferentially target solid tumors may selectively deliver a therapeutic agent to diseased tissue and release of particles from decaying isotopes can kill neighboring cells as well, and thereby may provide more complete tumor killing in poorly vascularized solid tumors. Another advantage of using transporter-targeted therapeutic agents comprising radioisotopes is that tumors in highly radiation sensitive organs such as the liver and pancreas may be selectively targeted.

Diagnostic agents of the present disclosure can be included in kits that may be administered to a patient to determine, for example, whether a patient has a disease that expresses a particular transporter, to determine whether a disease in a patient expresses a particular transporter, and/or to determine an absolute and/or a relative transporter expression level in diseased and/or non-diseased tissue in a patient. Diagnostic kits may include a plurality of diagnostic agents that are substrates for different transporters. A plurality of diagnostic agents may be adapted to assess the expression level of a broad array of transporters, or may be adapted to assess the expression of transporters known or believed to be expressed in a particular disease. For example, a plurality of diagnostic agents adapted to assess transporter expression levels in brain, lung, colon, and breast cancer may include diagnostic agents that are substrates for GLUT1, GLUT3, GLUT5, LAT1, ENT1, MCT1, MCT4, and SMVT. Diagnostic agents may be inherent substrates for a transporter or may be diagnostic conjugates, which comprise a diagnostic moiety and a substrate moiety. Each diagnostic conjugate included in a diagnostic kit can comprise the same or different diagnostic moiety, and the same or different substrate moiety. Diagnostic kits may include compositions comprising diagnostic agents that are transporter substrates and instructions for administering a diagnostically effective amount of the compositions to a patient. Compositions included in diagnostic kits may comprise a diagnostic agent that is a substrate for a transporter and a pharmaceutically acceptable vehicle.

Kits may be used to assess transporter expression in vivo. For in vivo uses, the diagnostic agents can be formulated into pharmaceutical compositions suitable for administration to a patient. In certain embodiments, kits may further include instructions for administering a pharmaceutical composition comprising a diagnostic agent to a patient in a manner and in an amount sufficient to determine if the patient has cells of a tissue associated with the disease characterized by at least a minimum predetermined expression level of at least one transporter expressed by the tissue.

Certain embodiments provided by the present disclosure include kits to be used for combined diagnosis and treatment. Such kits may include a first pharmaceutical composition comprising a diagnostic agent, a second pharmaceutical composition comprising a therapeutic agent, as well as instructions for administering the first pharmaceutical composition comprising a diagnostic agent to a patient in a manner and in an amount sufficient to determine if the patient has cells of a tissue associated with the disease characterized by at least a minimum predetermined expression level of at least one transporter expressed by the cells of the tissue and instructions for administering a therapeutically effective amount of the second pharmaceutical composition to the patient if it is determined that the patient has cells of a tissue associated with the disease characterized by at least the minimum predetermined expression level of the at least one transporter expressed by the cells of the tissue. In certain embodiments, cells of a diseased tissue or tissue associated with a diseased tissue have a variable transporter expression profile across a population of patients having the disease. Diagnostic compositions may include a diagnostic agent and a pharmaceutically acceptable vehicle, and a therapeutic composition may comprise a therapeutic agent and a pharmaceutically acceptable vehicle. A diagnostic agent and a therapeutic agent may be substrates for the same transporter or substrates for different transporters. In some applications, the diagnostic agent may be used to establish the presence of a disease in which cells associated with the disease express a transporter, and/or to determine an absolute or relative level of transporter expression in cells associated with the disease. Analysis of the disposition or transporter expression level as determined using the diagnostic agent within tissue following administration to the patient may then be used to determine whether to administer the composition comprising the therapeutic agent, or to determine a treatment regimen using the therapeutic agent. In certain embodiments, both the diagnostic agent and the therapeutic agent may be conjugates in which either a diagnostic moiety or a therapeutic moiety is bonded to a transporter substrate moiety. A transporter substrate moiety bonded to a diagnostic conjugate or to a therapeutic conjugate may have the same or different chemical structure. In certain embodiments, kits may be used to determine a transporter expression profile in cancerous tissue, and the second composition may comprise a therapeutic agent, which may be an antineoplastic agent.

Certain embodiments provided by the present disclosure include diagnostic kits for selecting a therapeutic agent for treating a disease in a patient, cells of a tissue associated with the disease having a variable transporter expression profile across a population of patients having the disease. Diagnostic kits may comprise a first diagnostic composition comprising a first diagnostic agent that is a substrate for a first transporter and a pharmaceutically acceptable vehicle, and a second diagnostic agent that is a substrate for a second transporter and a pharmaceutically acceptable vehicle. In certain embodiments, diagnostic kits may include more than two diagnostic compositions comprising diagnostic agents. Diagnostic agents may be substrates for a first or second transporter, or other transporter. In certain embodiments, diagnostic kits may include a plurality of diagnostic compositions comprising diagnostic agents, each of the diagnostic agents being a substrate for a different transporter. In certain embodiments, at least one of the diagnostic compositions in a kit comprises a diagnostic agent that is a diagnostic conjugate having a diagnostic moiety and a substrate moiety. Diagnostic kits provided by the present disclosure may further include instructions for administering a diagnostically effective amount of first and second diagnostic compositions to a patient, for measuring the expression profiles of first and second transporters in cells of tissue of the patient associated with the disease, and for selecting an appropriate therapeutic agent for treating the disease based upon the measured expression profiles of the first and second transporter. For kits having more than two diagnostic compositions, diagnostic kits may include such instructions for administering and using each of the pharmaceutical compositions. In certain embodiments, at least one of the diagnostic agents in a kit, such as either the first or second diagnostic agents, can be a conjugate comprising a diagnostic moiety and a substrate moiety. Diagnostic kits may be used to select a therapeutic agent for treating any tissue associated with a disease in a patient, such as cancerous tissue, brain tissue, and CNS tissue, in which tissue associated with the disease has a variable transporter expression profile across a population of patients having a disease.

In certain embodiments, kits may include one or more diagnostic compositions and one or more therapeutic compositions. The accompanying instructions may provide directions for administering one or more diagnostic compositions before, after, or during a treatment regimen with one or more therapeutic compositions.

### EXAMPLES

The following examples describe in detail methods of characterizing transporter expression. It will be apparent to those of ordinary skill in the art that many modifications, both to materials and methods, may be practiced.

### Example 1

### Transporter Expression in Tumor Samples and Cancer Cell Lines

mRNA profiling of human tumors demonstrated that the level of transporter expression in tumor cells varies depending on the transporter and/or tumor type. Using translated BLAST searches of the sequenced human genome, the full complement of plasma membrane transporters for organic solutes including 250 transporters belonging to approximately 20 different gene families was identified. The transporters included known nutrient transporters for sugars, nucleosides, amino acids, metabolic intermediates, vitamins, and general xenobiotics, as well as several orphan transporters. Using moderately high-throughput 96-well based profiling methods and validated qPCR primers for the 250 human transporters, mRNA expression for the transporters in 75 primary tumor biopsy samples and 100 normal tissue biopsy samples was measured. Tissue samples were obtained from Ardais Corporation and included information regarding tumor grade, morphology, and clinical history. Tissue samples included stage 2 adenocarcinomas from colon, lung, breast, ovarian, and prostate cancers with less than 10% stromal, necrotic, or normal tissue, as well as 20 metastatic tumor samples from colon and breast metastases. Tumor transporter expression was compared to expression in normal tissue from the same organ when matched normal tissue was available. Colon cancer tumor samples were compared with a panel of 60 normal colonic biopsy samples obtained from routine endoscopic procedures.

An initial survey of transporter expression in solid tumors identified approximately 25 transporters in which the median expression level of GAPDH was at least 0.3% in at least two of colon, lung, breast, and ovary cancer (Table 2).

Transporter expression in tumors evaluated using immunochemistry methods also showed a range of expression levels. Rabbit polyclonal antibodies for transporters expressed in tumors were developed using GST-fusion proteins, and each was validated and tested for specificity using the cloned full-length transporters expressed in transfected COS cells. The panel of antibodies included most of the transporters identified by qPCR profiling. Tumor tissue arrays containing 50-200 tumor and matched normal tissue samples per slide were obtained from Ambion and Lifespan Technologies. Slides typically contained tumor biopsy samples reflecting a variety of stages and morphologies. Twelve of the twenty antibodies were evaluated in colon, lung, and breast tumor tissue arrays. Several of the transporter antibodies were also evaluated in ovarian and glioma tumors. Immunohistochemistry was performed using standard protocols including epitope retrieval and o-diacetylbenzene (DAB) visualization. To quantify expression across hundreds of samples, staining was scored blind on a scale of 0-4 based on staining intensity and fraction of cells positive for transporter expression. Immunohistochemical staining results are also provided in Table 2 (Protein Levels).

TABLE 2
Summary of transporter mRNA expression in human tumor
biopsy samples (breast, lung, colon, and ovary)†

| | | mRNA level (% GAPDH) | | | | Protein Levels | | |
|---|---|---|---|---|---|---|---|---|
| Substrates | SLC | Bre | Lun | Col | Ova | Bre | Lun | Col |
| Sugars | GLUT1 | 1.8 | 1.4 | 1.2 | 2.1 | O | O | O |
| | GLUT3 | 0.3 | 0.8 | 0.0 | 0.5 | O | O | O |
| Mono- | MCT1 | 2.0 | 0.8 | 2.2 | 0.5 | O | O | O |
| carboxylates | MCT4 | 0.6 | 0.4 | 0.2 | 1.6 | O | O | O |
| Amino Acids | LAT1 | 0.2 | 0.7 | 1.2 | 0.2 | L | O | O |
| | F | 0.5 | 0.3 | 0.2 | 0.5 | L | L | L |
| | G | 0.9 | 0.8 | 0.5 | 0.8 | — | — | — |
| | H | 2.8 | 3.7 | 3.1 | 4.3 | H | H | H |
| | I | 3.2 | 1.6 | 2.2 | 2.2 | — | — | — |
| | J | 2.5 | 3.2 | 2.3 | 2.2 | — | — | — |
| | K | 0.2 | 1.0 | 0.2 | 0.0 | — | — | — |
| | L | 2.3 | 2.8 | 1.8 | 0.9 | — | — | — |
| | M | 0.3 | 0.3 | 0.2 | 0.7 | — | — | — |
| | N | 0.2 | 0.8 | 0.8 | 0.6 | — | — | — |
| | O | 0.6 | 2.6 | 0.9 | 1.0 | — | — | — |
| Nucleosides | ENT-1 | 1.6 | 2.4 | 1.9 | 2.1 | H | H | H |
| | Q | 0.3 | 0.6 | 0.5 | 0.2 | L | O | L |
| Organic | R | 0.5 | 1.8 | 0.6 | 1.2 | L | O | O |
| anions | S | 0.4 | 1.3 | 0.5 | 1.1 | — | — | — |
| | T | 0.1 | 1.0 | 1.3 | 0.6 | — | — | — |
| | U | 0.1 | 0.9 | 0.4 | 0.1 | L | O | — |
| Other | SMVT | 1.1 | 0.5 | 1.3 | 0.8 | O | O | O |
| | W | 0.2 | 0.6 | 0.1 | 0.3 | — | — | — |
| | X | 0.2 | 0.5 | 0.1 | 0.3 | O | L | L |
| | V | 0.3 | 0.3 | 0.2 | 0.1 | — | — | — |

†mRNA abundance is expressed as a percentage of GAPDH. Results for transporter protein staining in tumor tissue arrays are summarized in the last three columns:
O = higher in tumor than normal;
H = high in tumor and normal;
L = low in tumor and normal;
“—” = not tested.

### Example 2

### Quantitative PCR Detection of Transporter Expression in Tumor Cells

To measure the level of transporter expression in human tumors quantitative PCR was performed on human tumor mRNA obtained from Ardais Corporation. For comparison with normal colon, human colon mucosal tissue was obtained from endoscopy procedures. Table 3 shows high levels of GLUT1, GLUT3, GLUT5, LAT1, ENT1, and SMVT mRNA in human tumors.

Intestinal biopsy samples were obtained, with patient consent, from routine endoscopies or colonoscopies. Biopsies were taken from healthy sites by Radial Jaw 3 single use biopsy forceps (Boston Scientific) within the endoscope working channel. Each sample was approximately 3 mm in size. Samples were placed in numbered cryovials and snap frozen in liquid nitrogen. Vials were stored at −80° C. Biopsies were taken from up to three sites from a single patient.

Total RNA was isolated from all samples using the RNeasy RNA Isolation Kit (Qiagen). 1500 μl RLT Lysis Buffer+1% β-me was added to each biopsy. Samples were homogenized with a Powergen 125 Tissue Homogenizer (Fischer Scientific). Lysates were run though a Qiashredder column prior to RNA isolation (Qiagen). Total RNA was isolated by the procedures provided in the RNeasy RNA Isolation Manual. RNA was quantified and analyzed by agarose gel chromatography to ensure RNA integrity.

To synthesize single-stranded cDNA, one microgram of DNAseI treated total RNA (Invitrogen) was used as template per oligo dT primed Thermoscript RT reaction (Invitrogen). Following completion of cDNA synthesis, the RNA template was destroyed by RNAse H addition for 20 minutes at 37° C. To quantify the mRNA, single-stranded cDNA was amplified using transporter-specific primers in an MJ Research real-time PCR instrument using SYBR green fluorescent detection. The primer sequences used for quantitative PCR analysis of GLUT1, GLUT3, GLUT5, LAT1, LAT2, ENT1, and SMVT are provided in Table 4. Sample data was normalized using the mRNA abundance of GAPDH, and data shown in Table 3 indicates the number of mRNA transcripts in the quantitative PCR reaction.

These transporters exhibited higher mRNA levels in the tumor samples relative to normal tissues. Stage 2 colon adenocarcinoma samples exhibited more than about two-fold higher mRNA levels for the transporters GLUT1, GLUT3, GLUT5, LAT1, ENT1, and SMVT.

TABLE 3
Transporter mRNA Expression in Human
Stage 2 Adenocarcinomas

| | | | | | Tumor:Normal | |
|---|---|---|---|---|---|---|
| | mRNA Level-Stage 2 Adenocarcinoma | | | | Ratio | |
| | Breast | Lung | Colon | Ovary | | |
| | (11) | (11) | (15) | (4) | Colon | Ovary |
| GLUT1 | 11717 | 19364 | 10774 | 12009 | 1.6 | 9.7 |
| GLUT3 | 9695 | 24302 | 8391 | 25032 | 0.7 | 1.8 |
| GLUT5 | 862 | 2951 | 1097 | 3148 | 0.3 | 3.0 |
| LAT1 | 2192 | 4476 | 2062 | 2165 | 3.6 | 1.3 |
| ENT1 | 27125 | 24344 | 18687 | 52874 | 3.1 | 1.1 |
| SMVT | 13066 | 4722 | 10636 | 8294 | 5.5 | 4.1 |

TABLE 4

Primer Sequences Used for Quantitative PCR for
Transporters Expressed in Cancers

| | |
|---|---|
| Forward Primer | Reverse Primer |

| | | |
|---|---|---|
| hGLUT1 | ggggcatgattggctccttctctgtg | aggccgcagtacacaccgatgatgaa |
| | (SEQ ID NO: 1) | (SEQ ID NO:2) |
| hGLUT3 | tgtggtcctatgccgaatgccctcag | gcaccaagaagggaaagggagactga |
| | (SEQ ID NO: 3) | (SEQ ID NO: 4) |
| hGLUT5 | cccgagtcaattaaacagctggtcct | ggaagacctgttggggccaccgagtt |
| | (SEQ ID NO: 5) | (SEQ ID NO: 6) |
| hLAT1 | tgggacgtggacatgcctcaaggata | gggcacacagcaccaaggatcacaga |
| | (SEQ ID NO: 7) | (SEQ ID NO: 8) |
| hENT1 | ctgagtggtctggcggttttttcttc | acgcagacacacagacacatacacag |
| | (SEQ ID NO: 9) | (SEQ ID NO: 10) |
| hSMVT | cgaccttggttccctgagttctctga | ccaacaggcccacaacagcaccagga |
| | (SEQ ID NO: 11) | (SEQ ID NO: 12) |

### Example 3

### Staining of Tumor Samples

Immunohistochemical staining of tumor tissue microarrays enabled the expression patterns of transporters within tumor tissues to be examined. Antibodies that bind to transporters were developed and stained against a panel of human tumor samples. The results are summarized in Table 5.

A unique, relatively hydrophilic, sequence of amino acids was identified for GLUT1 (ASQSDKTPEELFHPLGADSQV) (SEQ ID NO: 13), GLUT3 (TRAFEGQAHGADRSGKDGVMEMNSIEPAKETTTNV) (SEQ ID NO: 14), GLUT5 (NQIFTKMNKVSEVYPEKEELKELPPVTSEQ) (SEQ ID NO: 15), LAT1 (MAGAGPKRRALAAPAAEEKEEAREKMLAAKSADGSAPAGEGEGVT) (SEQ ID NO: 16), ENT1 (QQLKLEGPGEQETKLDLISKGEEPRAGKEESGVSVSNSQPTNESHSIKAIL) (SEQ ID NO: 17), and SMVT (LSCQKRLHCRSYGQDHLDTGLFPEKPRNGVLGDSRDKEAMALDGTAYQGSSSTCILQETSL) (SEQ ID NO: 18)) using Vector NTI and BLAST analysis. Using PCR, this region of the transporter was amplified from cDNA using primers containing BamHI and EcoRI restriction sites to allow directional cloning into the GST-fusion vector pGEX-6P-1 (Amersham Biosciences). Constructs were sequenced and then placed into an IPTG inducible bacterial system to overexpress the GST-fusion protein. The protein was affinity purified and sent to CoCalico Biologicals, Inc. for polyclonal antibody production. COS-7 cells were transiently transfected with the indicated transporter or left untransfected as a control. Whole cell lysates were made and Western Blot analysis was performed using the affinity-purified polyclonal antibody. The antibodies were specific, and upon transfection, there was an increased signal of a protein band of the expected size. Some cross-reactivity with endogenous monkey transporter was observed.

Commercially available tumor tissue microarrays (Ambion) were used having the following characteristics: large sample size (50-250 tissues) per slide, matched benign controls, multiple types of tumors present on each slide, and clinical annotations for the various tissues.

The following staining procedure was used. Paraffin slides purchased from Ambion were baked for 1 hr at 37° C. and then for thirty minutes at 55° C. Tissues were then dewaxed with Biogenex EZ-DeWax solution as instructed by the manufacturer. Dewaxed slides were placed in an antigen retriever containing Retrievit Solution pH 8.0 (BioGenex). After briefly rinsing with water, tissues were blocked for endogenous peroxides with 3% hydrogen peroxide for 10 minutes. Slides were then rinsed with water and blocked with avidin followed by biotin for 15 minutes each. Non-specific binding was blocked by incubation in SuperBlock (PIERCE)+0.5% normal goat serum for 1 h. Slides were then incubated with primary antibody diluted in block for 1.5 h. Specimens were then rinsed three times for 5 minutes with PBS+0.1% Tween 20. Tissues were then incubated with biotinylated goat anti-rabbit immunoglobulins for 20 minutes, rinsed as above, and then incubated with streptavidin-horseradish peroxidase for 20 minutes. Slides were developed using DAB (diaminobenzidine) and hematoxylin as a nuclear counterstain. Tissues were covered with SuperMount (BioGenex) and then air-dried. The slides were examined using microscopy and scored for intensity of staining using a scale of zero to four (0 to 4), with a score of zero being the lightest staining (i.e., a staining that was similar to the staining achieved in the negative controls) and a score of four being the most heavily stained. Numbers in Table 5 are percentage transporter expression equal to or greater than 3 on the scale of 1-4 in various cancers.

TABLE 5
Transporter Expression in Tumors Determined by Immunohistochemical Staining

| | GLUT1 | GLUT3 | GLUT5 | LAT1 | ENT1 | SMVT |
|---|---|---|---|---|---|---|
| BRAIN CANCER | | | | | | |
| Astrocytoma (10) | 0* | 15 | 8 | 50 | — | — |
| Glioblastoma multiforme (28) | 21 | 18 | 19 | 38 | — | — |
| Normal Brain (11) | 9 | 0 | 0 | 72 | — | — |
| LUNG CANCER | | | | | | |
| Squamous cell carcinoma (10) | 63 | 50 | 0 | 45 | 53 | 40 |
| Adenocarcinoma (15) | 0 | 23 | 7 | 32 | 95 | 33 |
| Normal Lung (32) | 8 | 21 | 0 | 0 | 63 | 9 |
| COLON CANCER | | | | | | |
| Colon Adenocarcinoma (53) | 64 | 14 | 0 | 31 | 91 | 38 |
| Normal Colon | — | 0 | 12 | 0 | 50 | 30 |
| BREAST CANCER | | | | | | |
| Ductal Adenocarcinoma (36) | — | — | — | 1 | 89 | 61 |
| Normal Breast (18) | — | — | — | 0 | 0 | 22 |

*Percent.
Not measured.

### Example 4

### Quantitative PCR Detection of Transporter Expression in Brain Endothelial Cells

Quantitative PCR was performed to analyze transporter expression in human brain endothelial cells. Human brain tissue was obtained from epileptic foci surgically removed from human patients. Human brain microvessel endothelial cells were isolated using the following procedure. Brain tissues were washed in 70% ethanol and placed in sterile phosphate buffered saline. Meninges and surface vessels were removed. Cortical gray matter was minced, placed in preparation medium (1 g/L glucose, 25 mM HEPES, 100 U/ml penicillin, 100 μg/ml streptomycin, 10 μg/ml DNAse 1, 1 mg/ml collagenase/dispase, in DMEM, adjusted to a pH of 7.4), and incubated for 1 hour at 37° C. Samples were centrifuged for 10 minutes at 1000×g. Fat, cell debris, and myelin were discarded. The pellets were re-suspended in fresh preparation medium and incubated for an additional 3 hours at 37° C. in a shaking bath. Medium was filtered through a 230 μM nylon sieve followed by a 150 μM nylon sieve. Microvessels were collected by retention on a 60 μM nylon sieve. Capillaries were washed with preparation medium and then pelleted for RNA isolation.

Total RNA was isolated from the brain endothelial cells using the standard protocol for the RNEasy RNA Isolation Kit (Qiagen). Cells were re-suspended in RLT lysis buffer at 10 ml per 0.4 grams of cells. Lysates were vortexed and run through a QiaShredder Column (Qiagen) prior to RNA isolation. Once isolated, the RNA was quantified, run on a 1% agarose gel to ensure integrity, and then stored at −80° C.

Prior to cDNA synthesis, total RNA was DNAse I treated to destroy genomic DNA contamination (Invitrogen DNAseI Kit). Twenty microliters of oligo dT primed single-stranded cDNA was then synthesized from 1 μg total RNA (Invitrogen Thermoscript cDNA Synthesis Kit). The cDNA was treated with RNAse H and stored at −20° C.

Quantitative PCR was performed in a 96-well format using the MJ Research DNA Engine Opticon. For each transporter, a pair of 26-base oligonucleotide primers was used to amplify the specific transporter. Primers were designed to recognize the non-conserved 3′ ends of transporter mRNA. The single stranded cDNA was used as a template for a PCR reaction containing human, mouse or rat primers and SYBR Green master mix (Applied Biosystems). Fluorescent signal was read and graphed each cycle. A CT value, or cycle threshold value, was determined for each reaction. This value was defined as the point at which the fluorescent signal of the reaction exceeds background fluorescence. Background fluorescence was calculated as 20 standard deviations above the average signal from cycles 3 through 10. Transcript abundance was normalized to GAPDH transcript levels. Averaged results from 2 human transporter amplification experiments for the BBB transporters GLUT1, LAT1, MCT1, TAUT1, OAT3, OATPB, GAT2, OCT3, OCTN2, SVCT2, CAT1, and BGT1 are shown in Table 6.

TABLE 6
mRNA Expression in Human Brain Microvessel Endothelial Cells

| Transporter | Average BMEC | BMEC % GLUT1 | BMEC: Brain Ratio |
|---|---|---|---|
| GLUT1 | 802,859 | 100.0 | 43.1 |
| LAT1 | 114,571 | 14.3 | 19.1 |
| MCT1 | 169,370 | 21.1 | 13.9 |
| TAUT1 | 21,114 | 2.6 | 16.4 |
| OAT3 | 548 | 0.1 | 0 |
| OATPB | 218,903 | 27.3 | 28.8 |
| GAT2 | 29,104 | 3.6 | 14.3 |
| OCT3 | 10,330 | 1.3 | 1147.8 |
| OCTN2 | 11,708 | 1.5 | 3.9 |
| SVCT2 | 11,047 | 1.4 | 1.1 |
| CAT1 | 22,718 | 2.8 | 7.1 |
| BGT1 | 34,037 | 4.2 | 19.2 |

### Example 5

### Efficacy of a Transporter-Targeted Therapeutic Antineoplastic Agent

Bromopyruvate is described as a potent antineoplastic agent with demonstrated potential in animal models for treating glycolytic tumors such as hepatocellularcarcinoma (Ko et. al., Biochem, Biophys. Res. Commun. 2004, 324, 269-75; Geschwind et al., Cancer Res. 2002, 62, 3909-13). Bromopyruvate is an alkylating agent with structural similarity to natural substrates of MCT transporters, such as pyruvate.

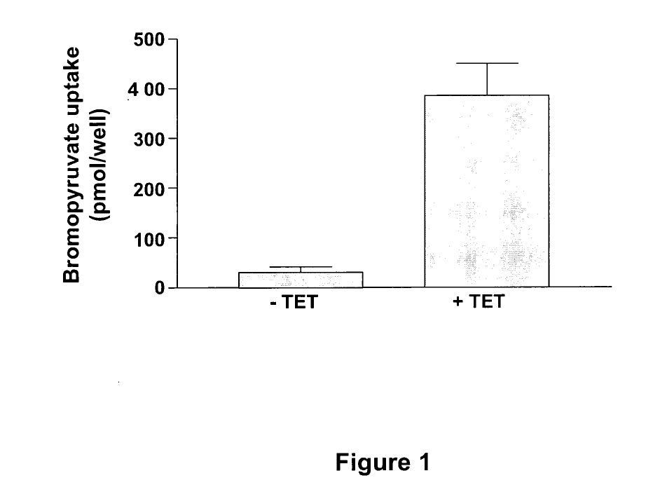

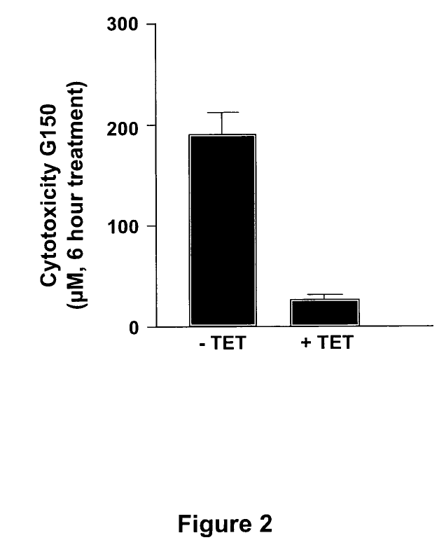

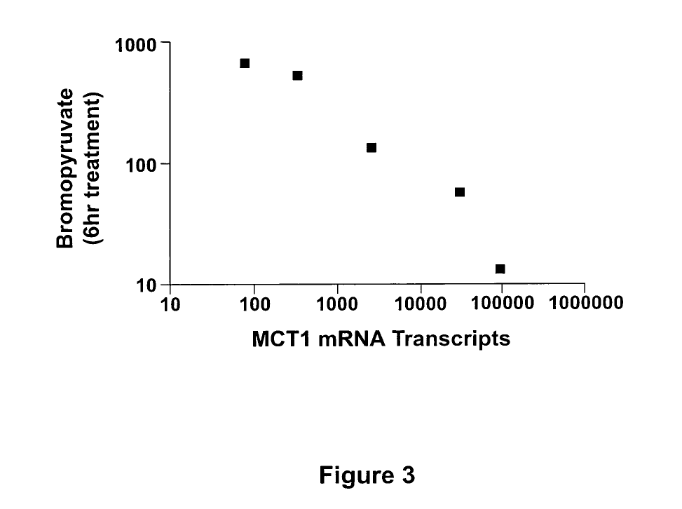

As shown in FIG. 1, bromopyruvate is accumulated in cells expressing MCT1 demonstrating that bromopyruvate is a true MCT1 substrate. Furthermore, as shown in FIG. 2, bromopyruvate is more than 20 times more toxic to cells that overexpress MCT1. The cytotoxicity of bromopyruvate was also determined for several cancer cell lines having varying levels of MCT1 expression. FIG. 3 shows that there is a strong correlation between the bromopyruvate GI50 and MCT1 mRNA levels. These results demonstrate that the expression level of an active transporter can have a significant effect on the potency of antineoplastic substrates.

### Example 6

### Phosphoramide Mustard Conjugates Targeted to Active Transporters

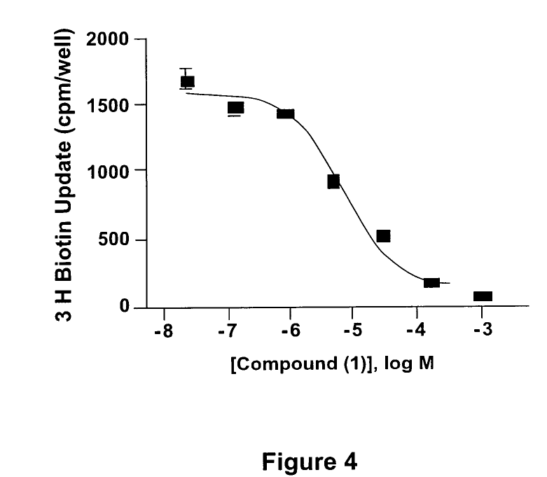

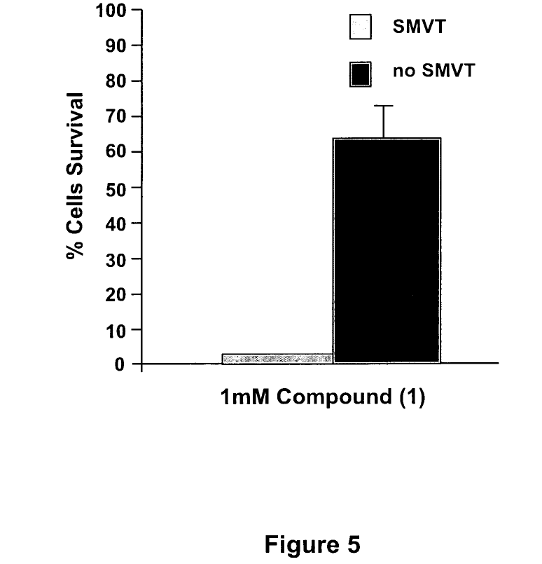

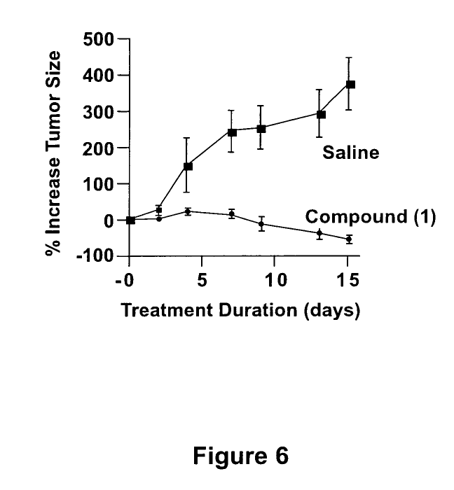

To demonstrate the feasibility of developing therapeutic conjugates, several phosphoramide mustards targeted to active transporters were synthesized. 4-({(acetyloxymethoxy)[bis(2-chloroethyl)amino]carbonyl}amino)butanoic acid (1) is a chemically stable phosphoramide prodrug. The active metabolite, bis(2-chlorethyl)amine, is released following cleavage of the phosphoester substrate moiety by intracellular carboxyesterases. Conjugate (1) exhibits good competitive inhibition of SMVT transport (FIG. 4) and electric currents when administered to Xenopus oocytes expressing SMVT (not shown), indicating that the conjugate is a transported substrate. The GI50 for conjugate (1) in HEK cells is shifted from 280 μM in parental cells to 18 μM in HEK cells overexpressing SMVT (FIG. 5). These results demonstrate that conjugate (1) is an SMVT substrate and the level of SMVT expression determines its antineoplastic potency. Conjugate (1) also showed good activity in a mouse tumor xenograft model (H69) (FIG. 6).

### Example 7

### Effect of Tumor Transporter Expression on Substrate Accumulation

Non-metabolized radiolabeled transporter substrates, ¹⁴C-1-methylglucose (GLUT1, GLUT3), ³H-biotin (SMVT), ¹⁴C-gabapentin (LAT1), ³H-gamma-hydroxybutyrate (MCT1), and ¹⁴C-formycin B (ENT1), are administered subcutaneously to tumor bearing mice. Radiolabeled non-transported compounds such as propranolol are used as negative controls. Tumors are removed after 20, 60, 120, and 240 minutes post-injection, homogenized, and the radiolabel content measured by scintillation counting. Kidney, liver, and plasma samples are also obtained and analyzed for comparison. Concentrative transporters such as SMVT and LAT1 can result in a more prolonged tumor accumulation than facilitative transporters such as GLUT1 and ENT1 that are expected to readily efflux the compounds as plasma levels decline. Increased transporter expression can result in more uptake of radiolabel into the tumors compared with the parental cell line. However, an increased transporter expression level should have no effect on the accumulation of the passively translocated control compounds.

Whole body autoradiography (WBA) is used to determine the disposition of actively transported substrates and the selectivity of substrate accumulation in tissue that expresses a transporter compared with other tissue. WBA allows both the visualization and the quantification of radiolabeled compound levels in a thin section of the whole animal. Information obtained using WBA is analogous to data obtained from diagnostic imaging, albeit at a single point in time.

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not as a limitation on the scope of the claims.

All publications and patents disclosed herein are incorporated by reference in their entirety.

What is claimed is:

1. A method of treating a disease in a patient, cells of a tissue of a patient associated with the disease having a variable expression profile for a transporter across a population of patients having the disease, the method comprising:
screening the cells of the tissue to determine whether the cells express the transporter at an expression level above a predetermined level;
if the cells of the tissue express the transporter above the predetermined level, then selecting a therapeutic agent from a plurality of therapeutic agents, each of the plurality of therapeutic agents being therapeutically effective for treating the disease, the selected therapeutic agent also being a substrate for the transporter; and
administering a therapeutically effective amount of the selected therapeutic agent to the patient.

2. The method of claim 1, wherein the screening comprises:
administering a diagnostic agent to the patient, wherein the diagnostic agent is a substrate for the transporter;
measuring the uptake of the diagnostic agent into the cells of the tissue; and
evaluating the measured uptake of the diagnostic agent to determine whether the cells of the tissue express at least the predetermined level of the transporter.

3. The method of claim 2, wherein the diagnostic agent is capable of producing an image and the measuring of the uptake of the diagnostic agent comprises recording an image of the cells of the tissue after administering the diagnostic agent to the patient.

4. The method of claim 1, wherein the screening comprises:
administering a diagnostic agent to the patient, wherein the diagnostic agent is a substrate for the transporter;
measuring transport of the diagnostic agent through the cells of the tissue; and
evaluating the measured transport of the diagnostic agent to determine whether the cells of the tissue express at least the predetermined level of the transporter.

5. The method of claim 4, wherein the diagnostic agent is capable of producing an image and the measuring of the transport of the diagnostic agent comprises recording an image of cells that the diagnostic agent is transported into after the transport through the cells of the tissue and after administering the diagnostic agent to the patient.

6. The method of claim 1, wherein the selected therapeutic agent is a therapeutic conjugate comprising a therapeutic moiety and a substrate moiety.

7. A method of determining an expression level of a transporter in cells of a tissue of a patient associated with a disease in a patient, the cells of the tissue associated with the disease having a variable expression profile for the transporter across a population of patients having the disease, the method comprising:
administering a diagnostic conjugate to the patient, wherein the diagnostic conjugate is a substrate for the transporter;
measuring the uptake of the diagnostic agent into the cells of the tissue; and
evaluating the measured uptake of the diagnostic conjugate to determine the expression level of the transporter in the cells of the tissue.

8. A method of determining an expression level of a transporter in cells of a first tissue associated with a disease in a patient, the method comprising:
administering a diagnostic agent to the patient, wherein the diagnostic agent is a substrate for the transporter;
measuring transport of the diagnostic agent through the cells of the first tissue; and
evaluating the measured transport of the diagnostic agent to determine the expression level of the transporter in the cells of the first tissue after administering the diagnostic agent to the patient.

9. The method of claim 8, wherein the cells of the first tissue associated with the disease have a variable expression profile for the transporter across a population of patients having the disease.

10. The method of claim 8, wherein the diagnostic agent is capable of producing an image and the measuring of the transport of the diagnostic agent comprises recording an image of cells of a second tissue that the diagnostic agent is transported into after the transport through the cells of the first tissue and after administering the diagnostic agent to the patient.

11. A method of determining the presence of a disease in a patient, the disease being characterized by an atypical level of expression for a transporter in cells of a first tissue of the patient associated with the disease, the method comprising:
measuring a first expression level of the transporter in cells of the first tissue of the patient; and
comparing the first expression level to a second expression level of the transporter, the second expression level being selected from:
(i) an expression level of the transporter measured in cells of a second tissue of the patient that is known to be free of the disease;
(ii) an expression level of the transporter measured in cells of a second tissue of the patient that is known to be unaffected by the presence of the disease;
(iii) an expression level of the transporter measured in cells of a tissue of another patient who is free of the disease; and
(iv) an expression level derived from a multiplicity of patients who are free of the disease.

12. The method of claim 11, wherein
the expression level of the transporter in the cells of the first tissue is atypical if the first and second transporter expression levels differ by more than a predetermined value; and
an atypical expression level of the transporter in the cells of the first tissue indicates the presence of the disease in the first tissue.

13. The method of claim 11, wherein the measuring of the first expression level comprises administering a diagnostic agent to the patient, the diagnostic agent being a substrate for the transporter.

14. The method of claim 11, further comprising administering a therapeutically effective amount of a therapeutic agent to the patient, the therapeutic agent being therapeutically effective for treating the disease and a substrate for the transporter.

15. A method of determining whether a disease in a patient is suitable to be treated with a therapeutic agent that is a substrate for a transporter, cells of a tissue of the patient associated with the disease having a variable expression profile for the transporter across a population of patients having the disease, the method comprising:
measuring an expression level of the transporter in cells of a tissue of the patient known to be associated with the disease;
comparing the measured expression level of the transporter with a minimum predetermined expression level known to be useful for the therapeutic agent to be effective in treating the disease; and
determining that the disease is suitable to be treated with the therapeutic agent if the measured expression level is at least the minimum predetermined expression level.

16. A method of monitoring treatment of a disease in a patient, the disease being characterized by an atypical expression level for a transporter in cells of a tissue of the patient associated with the disease, the method comprising:
measuring a first expression level in the cells of the tissue of the patient associated with the disease;
measuring a second expression level in the cells of the tissue of the patient associated with the disease, the second expression level being measured after the measuring of the first expression level and after a regimen of the treatment; and
comparing the second expression level with (i) or a combination of (i) and at least one of (ii)(a), (ii)(b), (ii)(c), and (ii)(d):
(i) the first expression level; and
(ii)(a) an expression level of the transporter measured in cells of a second tissue of the patient that is known to be free of the disease;
(ii)(b) an expression level of the transporter measured in cells of a second tissue of the patient that is known to be unaffected by the presence of the disease;
(ii)(c) an expression level of the transporter measured in cells of a tissue of another patient who is free of the disease; and
(ii)(d) an expression level of the transporter derived from a multiplicity of patients who are free of the disease.

17. The method of claim 16, wherein measuring the first and second expression levels comprises administering a diagnostic agent to the patient, wherein the diagnostic agent is a substrate for the transporter.

18. The method of claim 16, wherein the regimen of treatment comprises administering a therapeutic agent to the patient, the therapeutic agent being therapeutically effective for treating the disease and a substrate for the transporter.

19. A kit comprising:
a first composition comprising a diagnostic conjugate and a pharmaceutically acceptable vehicle, the diagnostic conjugate being a substrate for at least one transporter expressed by cells of a tissue of the patient associated with a disease; and
instructions for administering the first composition to the patient in a manner and in an amount sufficient to determine if the cells of the tissue of the patient associated with the disease are characterized by at least a minimum predetermined expression level of the at least one transporter.

20. The kit of claim 19, further comprising:
a second composition comprising a therapeutic agent that is a substrate for the at least one transporter and a pharmaceutically acceptable vehicle; and
instructions for administering a therapeutically effective amount of the second composition to the patient if it is determined that the patient has cells of a tissue associated with the disease characterized by at least the minimum predetermined expression level of the at least one transporter.

21. The kit of claim 20, wherein the therapeutic agent is a therapeutic conjugate comprising a therapeutic moiety and a substrate moiety.

22. A diagnostic kit for selecting a therapeutic agent for treating a disease in a patient, cells of a tissue associated with the disease having a variable expression profile for a first transporter across a population of patients having the disease, the diagnostic kit comprising:
a first diagnostic composition comprising a first diagnostic agent that is a substrate for a first transporter and a pharmaceutically acceptable vehicle;
a second diagnostic composition comprising a second diagnostic agent that is a substrate for a second transporter and a pharmaceutically acceptable vehicle; and
instructions for administering a diagnostically effective amount of the first and second diagnostic compositions to the patient for measuring the expression profiles of the first and second transporters in cells of the tissue of the patient associated with the disease, and for selecting an appropriate therapeutic agent for treating the disease based upon the measured expression profiles of the first and second transporters.
